(19) United States
(12) Patent Application Publication
(10) Pub. No.: US 2025/0072872 A1
(43) Pub. Date: Mar. 6, 2025

(54) PATHWAY MODIFICATION FOR COREGISTRATION OF EXTRALUMINAL IMAGE AND INTRALUMINAL DATA

(71) Applicant: PHILIPS IMAGE GUIDED THERAPY CORPORATION, SAN DIEGO, CA (US)

(72) Inventors: Ehud NACHTOMY, HERZLIYA (IL); Emily WINKLER BROWN, AMSTERDAM (NL); Michael ZARKH, NETANIA (IL)

(21) Appl. No.: 18/290,166

(22) PCT Filed: May 7, 2022
(86) PCT No.: PCT/EP2022/062384

§ 371 Date: Nov. 10, 2023

Related U.S. Application Data
(60) Provisional application No. 63/187,964, filed on May 13, 2021.

(51) Int. Cl. A61B 8/08; A61B 6/12; A61B 8/00; A61B 8/12; A61B 90/00; A61M 25/01

(52) U.S. Cl. CPC A61B 8/5261; A61B 6/12; A61B 8/12; A61B 8/463; A61B 2090/3966; A61M 25/0108

(57) ABSTRACT

A co-registration system includes a processor circuit that corrects the location of an indicator identifying a radiopaque portion of an intravascular catheter shown in an x-ray image and corrects the shape of a pathway defined by the movement of the radiopaque portion. The processor circuit receives, from the x-ray imaging device, x-ray images of a blood vessel while the intravascular catheter/guidewire moves through the blood vessel. The processor circuit receives, from the catheter, intravascular data representative of the blood vessel while the catheter moves through the blood vessel. The processor circuit co-registers the intravascular data to an x-ray image received from the x-ray imaging device. The processor circuit also displays the location indicator identifying the location of the radiopaque portion of the catheter in each x-ray image. In response to a user input, the processor circuit moves the location indicator to a corrected location. The processor circuit also modifies the shape of the pathway of the catheter in response to a user input. The processor circuit then completes the co-registration process again after either of these changes are made.

### TECHNICAL FIELD

The present disclosure relates generally to improving the accuracy of locations of intraluminal data coregistered within extraluminal images. In particular, locations of intravascular data may be adjusted and verified for accuracy within extraluminal fluoroscopy images after an imaging procedure.

### BACKGROUND

Physicians use many different medical diagnostic systems and tools to monitor a patient's health and diagnose and treat medical conditions. Different modalities of medical diagnostic systems may provide a physician with different images, models, and/or data relating to internal structures within a patient. These modalities include invasive devices and systems, such as intravascular systems, and non-invasive devices and systems, such as external ultrasound systems or x-ray systems. Using multiple diagnostic systems to examine a patient's anatomy provides a physician with added insight into the condition of the patient.

In the field of intravascular imaging and physiology measurement, co-registration of data from invasive devices (e.g. intravascular ultrasound (IVUS) devices) with images collected non-invasively (e.g. via x-ray angiography and/or x-ray venography) is a powerful technique for improving the efficiency and accuracy of vascular catheterization procedures. Co-registration identifies the locations of intravascular data measurements along a blood vessel by mapping the data to an x-ray image of the vessel. A physician may then see on an angiography image exactly where along the vessel a measurement was made, rather than estimate the location.

When intravascular measurements are co-registered to locations within an angiography image, errors may occur. Co-registration systems may show an intravascular data point in a location distal or proximal to the actual measurement location along the imaged vessel. Sometimes systems may also show an intravascular data point at a location not along the imaged vessel, but at some distance adjacent to the vessel. These errors in the locations of intravascular measurements may affect a physician's ability to properly diagnose and treat the medical condition and may sometimes require a patient to undergo additional intravascular procedures.

### SUMMARY

Embodiments of the present disclosure are systems, devices, and methods for improving the accuracy of the coregistered locations of intraluminal data within an extraluminal fluoroscopy image. For example, the intraluminal data can be intravascular imaging data, such as intravascular ultrasound (IVUS) images obtained with an intravascular device. The extraluminal images can be x-ray images. In an imaging procedure, the intravascular device is moved through a vessel of the patient acquiring IVUS images while x-ray images are also obtained showing the same vessel of the patient. A processor circuit then receives the x-ray images and uses image processing techniques to identify the location of the intravascular device in each x-ray image. The processor circuit then compiles the locations of the intravascular device to generate a pathway and assigns each IVUS image to a location along that pathway. The pathway identifying the locations of IVUS images may be overlaid on an x-ray image and displayed to a user.

In one aspect, the user may view all of the x-ray images obtained during the imaging procedure in a continuous image stream. Throughout the image stream, the processor circuit displays to the user its determined location of the intravascular device as it moves through the patient's vessel with an indicator that moves along with the device within each x-ray image. If the user observes that the location of the indicator is different from the location of the intravascular device, the user may pause the image stream and move the indicator to match the location of the intravascular device. After the user makes this correction, the system recalculates all the locations of IVUS images within the x-ray image based on the corrected location.

In another aspect, the user may view the pathway overlaid on an x-ray image and observe that the pathway is not the same shape as the vessel as shown by a guidewire inserted within the vessel. The user then adjusts the shape of the pathway to match the shape of the guidewire within the x-ray image. The system may then, again, recalculate the locations of IVUS images within the x-ray image based on the corrected pathway shape.

In an exemplary aspect, a system is provided. The system comprises a processor circuit configured for communication with an extraluminal imaging device, wherein the processor circuit is configured to receive a plurality of extraluminal images obtained by the extraluminal imaging device during movement of an intraluminal catheter within a body lumen of a patient, wherein the plurality of extraluminal images show a radiopaque portion of the intraluminal catheter; determine a location of the radiopaque portion in a first extraluminal image of the plurality of extraluminal images; output, to a display in communication with the processor circuit, a first screen display comprising the first extraluminal image; and a first marking in the first extraluminal image representative of the determined location of the radiopaque portion; receive a user input comprising a corrected location of the radiopaque portion; and output, to the display, a second screen display comprising the first extraluminal image; and a second marking in the first extraluminal image representative of the corrected location.

In one aspect, the processor circuit is configured to determine a corresponding location of the radiopaque portion in each extraluminal image of a plurality of extraluminal images; output, to the display, the plurality of extraluminal images such that a respective extraluminal image in the first screen display includes the first marking representative of the corresponding location of the radiopaque portion. In one aspect, the plurality of extraluminal images show the radiopaque portion at different positions corresponding to the movement of the intraluminal catheter. In one aspect, the plurality of extraluminal images show the first marking at different positions corresponding to the movement of the intraluminal catheter. In one aspect, the processor circuit is configured to receive a further user input comprising a selection of the first extraluminal image from among the plurality of extraluminal images. In one aspect, the processor circuit is configured to output, to the display, the plurality of extraluminal images such that the first extraluminal image in the second screen display includes the second marking representative of the corrected location. In one aspect, the processor circuit is configured to determine a path of the movement based on the corresponding locations of the radiopaque portion in the plurality of extraluminal images, wherein the first extraluminal image in first screen display further includes the determined path. In one aspect, the intraluminal catheter comprises an intraluminal imaging catheter, wherein the processor circuit is configured for communication with the intraluminal imaging catheter, wherein the processor circuit is configured to receive a plurality of intraluminal images obtained by the intraluminal imaging catheter during the movement of the intraluminal imaging catheter; and coregister the plurality of intraluminal images to corresponding positions along the determined path. In one aspect, the processor circuit is configured to determine a corrected position along the determined path for a corresponding intraluminal image based on the user input comprising the corrected location of the radiopaque portion. In one aspect, the processor circuit is configured to output, to the display, a third screen display comprising a second extraluminal image of the plurality of extraluminal images; the corresponding intraluminal image; and a third marking in the extraluminal image representative of the corrected position of the intraluminal image along the path. In one aspect, the processor circuit is configured to further coregister the plurality of intraluminal images to corresponding positions along the determined path based on the user input comprising the corrected location of the radiopaque portion. In one aspect, the movement of the intraluminal catheter is along a guidewire within the body lumen, wherein the plurality of extraluminal images further show the guidewire, wherein the path matches the shape of the guidewire within the body lumen. In one aspect, the plurality of extraluminal images are obtained without a contrast agent within the blood vessel.

In an exemplary aspect, a system is provided. The system comprises an intravascular ultrasound (IVUS) imaging catheter; and a processor circuit configured for communication with an x-ray imaging device and the IVUS catheter, wherein the processor circuit is configured to receive a plurality of x-ray images obtained by the x-ray imaging device during movement of the IVUS imaging catheter within a blood vessel of a patient, wherein the plurality of x-ray images are obtained without a contrast agent within the blood vessel, wherein IVUS plurality of extraluminal images show a radiopaque portion of the intraluminal catheter; receive a plurality of IVUS images obtained by the IVUS imaging catheter during the movement of the IVUS imaging catheter; determine a path of the movement based on corresponding locations of the radiopaque portion in the plurality of x-ray images; co-register the plurality of IVUS images to corresponding positions along the path; output, to a display in communication with the processor circuit, a first screen display comprising the plurality of x-ray images; and a first marking in each x-ray image of the plurality of x-ray images representative of the corresponding location of the radiopaque portion such that the first marking is shown at different positions corresponding to the movement of the intraluminal catheter; receive a user input comprising a corrected location of the radiopaque portion in a first x-ray image of the plurality of x-ray images; determine a corrected position along the determined path for a corresponding IVUS image based on the user input comprising the corrected location of the radiopaque portion; and output, to the display, a second screen display comprising a second x-ray image of the plurality of x-ray images; the corresponding IVUS image; and a second marking in the second x-ray image representative of the corrected position along the path.

In an exemplary aspect, a system is provided. The system comprises a processor circuit configured for communication with an extraluminal imaging device, wherein the processor circuit is configured to receive a plurality of extraluminal images obtained by the extraluminal imaging device during movement of an intraluminal catheter along a guidewire within a body lumen of a patient, wherein the plurality of extraluminal images are obtained without a contrast agent within the body lumen, wherein the plurality of extraluminal images show the guidewire and a radiopaque portion of the intraluminal catheter; determine a path of the movement based on corresponding locations of the radiopaque portion in the plurality of extraluminal images, wherein the path comprises a first shape; output, to a display in communication with the processor circuit, a first screen display comprising an extraluminal image of the plurality of extraluminal images; and the path in the extraluminal image with the first shape; receive a user input comprising a second shape of the path, wherein the second shape matches a shape of the guidewire in the extraluminal image; and output, to the display, a second screen display comprising the extraluminal image; and the path in the extraluminal image with the second shape.

In an exemplary aspect, a system is provided. The system comprises an intravascular ultrasound (IVUS) imaging catheter; and a processor circuit configured for communication with an x-ray imaging device and the IVUS catheter, wherein the processor circuit is configured to receive a plurality of x-ray images obtained by the x-ray imaging device during movement of the IVUS imaging catheter along a guidewire within a blood vessel of a patient, wherein the plurality of x-ray images are obtained without a contrast agent within the blood vessel, wherein the plurality of extraluminal images show the guidewire and a radiopaque portion of the IVUS catheter; receive a plurality of IVUS images obtained by the IVUS imaging catheter during the movement of the IVUS imaging catheter; determine a path of the movement based on corresponding locations of the radiopaque portion in the plurality of x-ray images, wherein the path comprises a first shape; output, to a display in communication with the processor circuit, a first screen display comprising an x-ray image of the plurality of x-ray images; and the path in the x-ray image with the first shape; receive a user input comprising a second shape of the path, wherein the second shape matches a shape of the guidewire in the x-ray image; co-register the plurality of IVUS images to positions along the path with the second shape; and output, to the display, a second screen display comprising a second x-ray image of the plurality of x-ray images; an IVUS image; and a marking in the x-ray image representative a corresponding position of the IVUS image along the path with the second shape.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of an intraluminal imaging and x-ray system, according to aspects of the present disclosure.

FIG. 2 is a diagrammatic top view of an ultrasound imaging assembly in a flat configuration, according to aspects of the present disclosure.

FIG. 3 is a diagrammatic perspective view of the ultrasound imaging assembly shown in FIG. 2 in a rolled configuration around a support member, according to aspects of the present disclosure.

FIG. 4 is a diagrammatic cross-sectional side view of the ultrasound imaging assembly shown in FIG. 3, according to aspects of the present disclosure.

FIG. 5 is a schematic diagram of a processor circuit, according to aspects of the present disclosure.

FIG. 6 is a diagrammatic view of an x-ray fluoroscopy image illustrating a pullback procedure, according to aspects of the present disclosure.

FIG. 7 is a diagrammatic view of a relationship between x-ray fluoroscopy images, intravascular data, and a path defined by the motion of an intravascular device, according to aspects of the present disclosure.

FIG. 8 is a diagrammatic view of a graphical user interface displaying a coregistration pathway overlaid on an x-ray image, according to aspects of the present disclosure.

FIG. 9 is a diagrammatic view of a graphical user interface displaying a marker identifying a location of an imaging device within an x-ray image, according to aspects of the present disclosure.

FIG. 10 is a diagrammatic view of a graphical user interface displaying a marker incorrectly identifying a location of an imaging device within an x-ray image, according to aspects of the present disclosure.

FIG. 11 is a diagrammatic view of a graphical user interface displaying an IVUS image coregistered to an incorrect location within an x-ray image, according to aspects of the present disclosure.

FIG. 12 is a diagrammatic view of a graphical user interface displaying a marker moved to the correct location of an imaging device within an x-ray image, according to aspects of the present disclosure.

FIG. 13 is a diagrammatic view of a graphical user interface displaying an IVUS image coregistered to its correct location within an x-ray image, according to aspects of the present disclosure.

FIG. 14 is a diagrammatic view of a graphical user interface displaying a coregistration pathway with a portion that does not have the same shape as a guidewire, according to aspects of the present disclosure.

FIG. 15 is a diagrammatic view of a graphical user interface displaying a coregistration pathway that has been corrected to have the same shape as a guidewire, according to aspects of the present disclosure.

FIG. 16 is a flow diagram for a method of modification of the location of an intraluminal imaging device within an extraluminal image, according to aspects of the present disclosure.

FIG. 17 is a flow diagram for a method of pathway modification for coregistration of an extraluminal image and intraluminal data, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

The devices, systems, and methods described herein can include one or more features described in U.S. Provisional Application No. 63/187,962, filed May 13, 2021, and titled “Coregistration Reliability with Extraluminal Image and Intraluminal Data” (Atty Dkt No. 2021PF00090/44755.2198PV01), U.S. Provisional Application No. 63/187,983, filed May 13, 2021, and titled “Coregistration of Intraluminal Data to Guidewire in Extraluminal Image Obtained Without Contrast” (Atty Dkt No. 2021PF00092/44755.2200PV01), U.S. Provisional Application No. 63/187,990, filed May 13, 2021, and titled “Preview of Intraluminal Ultrasound Image Along Longitudinal View of Body Lumen” (Atty Dkt No. 2021PF00093/44755.2201PV01), and U.S. Provisional Application No. 63/187,961, filed May 13, 2021, and titled “Intraluminal Treatment Guidance from Prior Extraluminal Imaging, Intraluminal Data, and Coregistration” (Atty Dkt No. 2021PF00012/44755.2192PV01), each of which is incorporated by reference herein in its entirety.

The devices, systems, and methods described herein can also include one or more features described in European Application No. 21154591.8, filed Feb. 1, 2021, and titled “X-Ray and Intravascular Ultrasound Image Registration”, which is incorporated by reference herein in its entirety.

The devices, systems, and methods described herein can also include one or more features described in U.S. Publication No. 2020/0129144, titled “Disease Specific and Treatment Type Specific Control of Intraluminal Ultrasound Imaging”, U.S. Publication No. 2020/0129142, titled “Intraluminal Ultrasound Navigation Guidance and Associated Devices, Systems, And Methods”, U.S. Publication No. 2020/0129148, titled “Intraluminal Ultrasound Imaging with Automatic and Assisted Labels And Bookmarks”, U.S. Publication No. 2020/0129158, titled “Graphical Longitudinal Display for Intraluminal Ultrasound Imaging and Associated Devices, Systems, and Methods”, U.S. Publication No. 2020/0129147, titled “Intraluminal Ultrasound Vessel Border Selection and Associated Devices, Systems, and Methods”, U.S. Publication No. 2020/0129159, titled “Intraluminal Ultrasound Directional Guidance and Associated Devices, Systems, and Methods”, U.S. Publication No. 2020/0129143, titled “Speed Determination for Intraluminal Ultrasound Imaging and Associated Devices, Systems, And Methods”, each of which is incorporated by reference herein in its entirety.

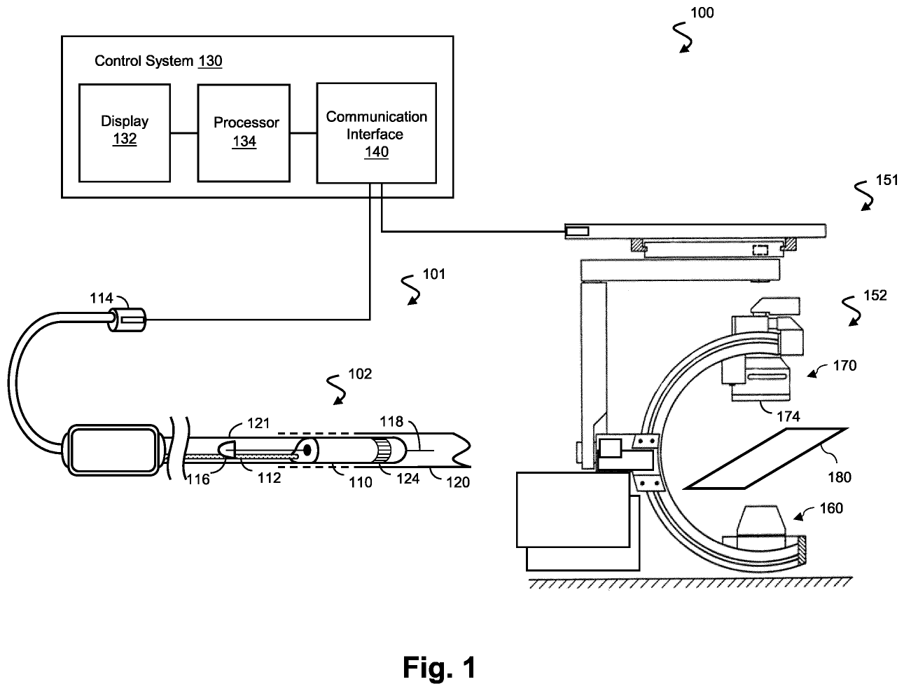

FIG. 1 is a schematic diagram of an intraluminal imaging and x-ray system 100, according to aspects of the present disclosure. In some embodiments, the intraluminal imaging and x-ray system 100 may include two separate systems or be a combination of two systems: an intraluminal sensing system 101 and an extraluminal imaging system 151. The intraluminal sensing system 101 obtains medical data about a patient's body while the intraluminal device 102 is positioned inside the patient's body. For example, the intraluminal sensing system 101 can control the intraluminal device 102 to obtain intraluminal images of the inside of the patient's body while the intraluminal device 102 is inside the patient's body. The extraluminal imaging system 151 obtains medical data about the patient's body while the extraluminal imaging device 152 is positioned outside the patient's body. For example, the extraluminal imaging system 151 can control extraluminal imaging device 152 to obtain extraluminal images of the inside of the patient's body while the extraluminal imaging device 152 is outside the patient's body.

The intraluminal imaging system 101 may be in communication with the extraluminal imaging system 151 through any suitable components. Such communication may be established through a wired cable, through a wireless signal, or by any other means. In addition, the intraluminal imaging system 101 may be in continuous communication with the x-ray system 151 or may be in intermittent communication. For example, the two systems may be brought into temporary communication via a wired cable, or brought into communication via a wireless communication, or through any other suitable means at some point before, after, or during an examination. In addition, the intraluminal system 101 may receive data such as x-ray images, annotated x-ray images, metrics calculated with the x-ray imaging system 151, information regarding dates and times of examinations, types and/or severity of patient conditions or diagnoses, patient history or other patient information, or any suitable data or information from the x-ray imaging system 151. The x-ray imaging system 151 may also receive any of these data from the intraluminal imaging system 101. In some embodiments, and as shown in FIG. 1, the intraluminal imaging system 101 and the x-ray imaging system 151 may be in communication with the same control system 130. In this embodiment, both systems may be in communication with the same display 132, processor 134, and communication interface 140 shown as well as in communication with any other components implemented within the control system 130.

In some embodiments, the system 100 may not include a control system 130 in communication with the intraluminal imaging system 101 and the x-ray imaging system 151. Instead, the system 100 may include two separate control systems. For example, one control system may be in communication with or be a part of the intraluminal imaging system 101 and an additional separate control system may be in communication with or be a part of the x-ray imaging system 151. In this embodiment, the separate control systems of both the intraluminal imaging system 101 and the x-ray imaging system 151 may be similar to the control system 130. For example, each control system may include various components or systems such as a communication interface, processor, and/or a display. In this embodiment, the control system of the intraluminal imaging system 101 may perform any or all of the coregistration steps described in the present disclosure. Alternatively, the control system of the x-ray imaging system 151 may perform the coregistration steps described.

The intraluminal imaging system 101 can be an ultrasound imaging system. In some instances, the intraluminal imaging system 101 can be an intravascular ultrasound (IVUS) imaging system. The intraluminal imaging system 101 may include an intraluminal imaging device 102, such as a catheter, guide wire, or guide catheter, in communication with the control system 130. The control system 130 may include a display 132, a processor 134, and a communication interface 140 among other components. The intraluminal imaging device 102 can be an ultrasound imaging device. In some instances, the device 102 can be an IVUS imaging device, such as a solid-state IVUS device.

At a high level, the IVUS device 102 emits ultrasonic energy from a transducer array 124 included in a scanner assembly, also referred to as an IVUS imaging assembly, mounted near a distal end of the catheter device. The ultrasonic energy is reflected by tissue structures in the surrounding medium, such as a vessel 120, or another body lumen surrounding the scanner assembly 110, and the ultrasound echo signals are received by the transducer array 124. In that regard, the device 102 can be sized, shaped, or otherwise configured to be positioned within the body lumen of a patient. The communication interface 140 transfers the received echo signals to the processor 134 of the control system 130 where the ultrasound image (including flow information in some embodiments) is reconstructed and displayed on the display 132. The control system 130, including the processor 134, can be operable to facilitate the features of the IVUS imaging system 101 described herein. For example, the processor 134 can execute computer readable instructions stored on the non-transitory tangible computer readable medium.

The communication interface 140 facilitates communication of signals between the control system 130 and the scanner assembly 110 included in the IVUS device 102. This communication includes the steps of: (1) providing commands to integrated circuit controller chip(s) included in the scanner assembly 110 to select the particular transducer array element(s), or acoustic element(s), to be used for transmit and receive, (2) providing the transmit trigger signals to the integrated circuit controller chip(s) included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s) of the scanner assembly 110. In some embodiments, the communication interface 140 performs preliminary processing of the echo data prior to relaying the data to the processor 134. In examples of such embodiments, the communication interface 140 performs amplification, filtering, and/or aggregating of the data. In an embodiment, the communication interface 140 also supplies high- and low-voltage DC power to support operation of the device 102 including circuitry within the scanner assembly 110.

The processor 134 receives the echo data from the scanner assembly 110 by way of the communication interface 140 and processes the data to reconstruct an image of the tissue structures in the medium surrounding the scanner assembly 110. The processor 134 outputs image data such that an image of the lumen 120, such as a cross-sectional image of the vessel 120, is displayed on the display 132. The lumen 120 may represent fluid filled or surrounded structures, both natural and man-made. The lumen 120 may be within a body of a patient. The lumen 120 may be a blood vessel, such as an artery or a vein of a patient's vascular system, including cardiac vasculature, peripheral vasculature, neural vasculature, renal vasculature, and/or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

In some embodiments, the IVUS device includes some features similar to traditional solid-state IVUS catheters, such as the EagleEye® catheter, Visions PV.014P RX catheter, Visions PV.018 catheter, Visions PV.035, and Pioneer Plus catheter, each of which are available from Koninklijke Philips N.V, and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors. It is understood that any suitable gauge wire can be used for the conductors. In an embodiment, the cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43 AWG gauge wires can be used.

The transmission line bundle 112 terminates in a patient interface module (PIM) connector 114 at a proximal end of the device 102. The PIM connector 114 electrically couples the transmission line bundle 112 to the communication interface 140 and physically couples the IVUS device 102 to the communication interface 140. In some embodiments, the communication interface 140 may be a PIM. In an embodiment, the IVUS device 102 further includes a guide wire exit port 116. Accordingly, in some instances the IVUS device 102 is a rapid-exchange catheter. The guide wire exit port 116 allows a guide wire 118 to be inserted towards the distal end to direct the device 102 through the vessel 120.

In some embodiments, the intraluminal imaging device 102 may acquire intravascular images of any suitable imaging modality, including optical coherence tomography (OCT) and intravascular photoacoustic (IVPA).

In some embodiments, the intraluminal device 102 is a pressure sensing device (e.g., pressure-sensing guidewire) that obtains intraluminal (e.g., intravascular) pressure data, and the intraluminal system 101 is an intravascular pressure sensing system that determines pressure ratios based on the pressure data, such as fractional flow reserve (FFR), instantaneous wave-free ratio (iFR), and/or other suitable ratio between distal pressure and proximal/aortic pressure (Pd/Pa). In some embodiments, the intraluminal device 102 is a flow sensing device (e.g., flow-sensing guidewire) that obtains intraluminal (e.g., intravascular) flow data, and the intraluminal system 101 is an intravascular flow sensing system that determines flow-related values based on the pressure data, such as coronary flow reserve (CFR), flow velocity, flow volume, etc.

The x-ray imaging system 151 may include an x-ray imaging apparatus or device 152 configured to perform x-ray imaging, angiography, fluoroscopy, radiography, venography, among other imaging techniques. The x-ray imaging system 151 can generate a single x-ray image (e.g., an angiogram or venogram) or multiple (e.g., two or more) x-ray images (e.g., a video and/or fluoroscopic image stream) based on x-ray image data collected by the x-ray device 152. The x-ray imaging device 152 may be of any suitable type, for example, it may be a stationary x-ray system such as a fixed c-arm x-ray device, a mobile c-arm x-ray device, a straight arm x-ray device, or a u-arm device. The x-ray imaging device 152 may additionally be any suitable mobile device. The x-ray imaging device 152 may also be in communication with the control system 130. In some embodiments, the x-ray system 151 may include a digital radiography device or any other suitable device.

The x-ray device 152 as shown in FIG. 1 includes an x-ray source 160 and an x-ray detector 170 including an input screen 174. The x-ray source 160 and the detector 170 may be mounted at a mutual distance. Positioned between the x-ray source 160 and the x-ray detector 170 may be an anatomy of a patient or object 180. For example, the anatomy of the patient (including the vessel 120) can be positioned between the x-ray source 160 and the x-ray detector 170.

The x-ray source 160 may include an x-ray tube adapted to generate x-rays. Some aspects of the x-ray source 160 may include one or more vacuum tubes including a cathode in connection with a negative lead of a high-voltage power source and an anode in connection with a positive lead of the same power source. The cathode of the x-ray source 160 may additionally include a filament. The filament may be of any suitable type or constructed of any suitable material, including tungsten or rhenium tungsten, and may be positioned within a recessed region of the cathode. One function of the cathode may be to expel electrons from the high voltage power source and focus them into a well-defined beam aimed at the anode. The anode may also be constructed of any suitable material and may be configured to create x-radiation from the emitted electrons of the cathode. In addition, the anode may dissipate heat created in the process of generating x-radiation. The anode may be shaped as a beveled disk and, in some embodiments, may be rotated via an electric motor. The cathode and anode of the x-ray source 160 may be housed in an airtight enclosure, sometimes referred to as an envelope.

In some embodiments, the x-ray source 160 may include a radiation object focus which influences the visibility of an image. The radiation object focus may be selected by a user of the system 100 or by a manufacture of the system 100 based on characteristics such as blurring, visibility, heat-dissipating capacity, or other characteristics. In some embodiments, an operator or user of the system 100 may switch between different provided radiation object foci in a point-of-care setting.

The detector 170 may be configured to acquire x-ray images and may include the input screen 174. The input screen 174 may include one or more intensifying screens configured to absorb x-ray energy and convert the energy to light. The light may in turn expose a film. The input screen 174 may be used to convert x-ray energy to light in embodiments in which the film may be more sensitive to light than x-radiation. Different types of intensifying screens within the image intensifier may be selected depending on the region of a patient to be imaged, requirements for image detail and/or patient exposure, or any other factors. Intensifying screens may be constructed of any suitable materials, including barium lead sulfate, barium strontium sulfate, barium fluorochloride, yttrium oxysulfide, or any other suitable material. The input screen 374 may be a fluorescent screen or a film positioned directly adjacent to a fluorescent screen. In some embodiments, the input screen 374 may also include a protective screen to shield circuitry or components within the detector 370 from the surrounding environment. In some embodiments, the x-ray detector 170 may include a flat panel detector (FPD). The detector 170 may be an indirect conversion FPD or a direct conversion FPD. The detector 170 may also include charge-coupled devices (CCDs). The x-ray detector 370 may additionally be referred to as an x-ray sensor.

The object 180 may be any suitable object to be imaged. In an exemplary embodiment, the object may be the anatomy of a patient. More specifically, the anatomy to be imaged may include chest, abdomen, the pelvic region, neck, legs, head, feet, a region with cardiac vasculature, or a region containing the peripheral vasculature of a patient and may include various anatomical structures such as, but not limited to, organs, tissue, blood vessels and blood, gases, or any other anatomical structures or objects. In other embodiments, the object may be or include man-made structures.

In some embodiments, the x-ray imaging system 151 may be configured to obtain x-ray images without contrast. In some embodiments, the x-ray imaging system 151 may be configured to obtain x-ray images with contrast (e.g., angiogram or venogram). In such embodiments, a contrast agent or x-ray dye may be introduced to a patient's anatomy before imaging. The contrast agent may also be referred to as a radiocontrast agent, contrast material, contrast dye, or contrast media. The contrast dye may be of any suitable material, chemical, or compound and may be a liquid, powder, paste, tablet, or of any other suitable form. For example, the contrast dye may be iodine-based compounds, barium sulfate compounds, gadolinium-based compounds, or any other suitable compounds. The contrast agent may be used to enhance the visibility of internal fluids or structures within a patient's anatomy. The contrast agent may absorb external x-rays, resulting in decreased exposure on the x-ray detector 170.

In some embodiments, the extraluminal imaging system 151 could be any suitable extraluminal imaging device, such as computed tomography (CT) or magnetic resonance imaging (MRI).

When the control system 130 is in communication with the x-ray system 151, the communication interface 140 facilitates communication of signals between the control system 130 and the x-ray device 152. This communication includes providing control commands to the x-ray source 160 and/or the x-ray detector 170 of the x-ray device 152 and receiving data from the x-ray device 152. In some embodiments, the communication interface 140 performs preliminary processing of the x-ray data prior to relaying the data to the processor 134. In examples of such embodiments, the communication interface 140 may perform amplification, filtering, and/or aggregating of the data. In an embodiment, the communication interface 140 also supplies high- and low-voltage DC power to support operation of the device 152 including circuitry within the device.

The processor 134 receives the x-ray data from the x-ray device 152 by way of the communication interface 140 and processes the data to reconstruct an image of the anatomy being imaged. The processor 134 outputs image data such that an image is displayed on the display 132. In an embodiment in which the contrast agent is introduced to the anatomy of a patient and a venogram is to be generated, the particular areas of interest to be imaged may be one or more blood vessels or other section or part of the human vasculature. The contrast agent may identify fluid filled structures, both natural and/or man-made, such as arteries or veins of a patient's vascular system, including cardiac vasculature, peripheral vasculature, neural vasculature, renal vasculature, and/or any other suitable lumen inside the body. For example, the x-ray device 152 may be used to examine any number of anatomical locations and tissue types, including without limitation all the organs, fluids, or other structures or parts of an anatomy previously mentioned. In addition to natural structures, the x-ray device 152 may be used to examine man-made structures such as any of the previously mentioned structures.

The processor 134 may be configured to receive an x-ray image that was stored by the x-ray imaging device 152 during a clinical procedure. The images may be further enhanced by other information such as patient history, patient record, IVUS imaging, pre-operative ultrasound imaging, pre-operative CT, or any other suitable data.

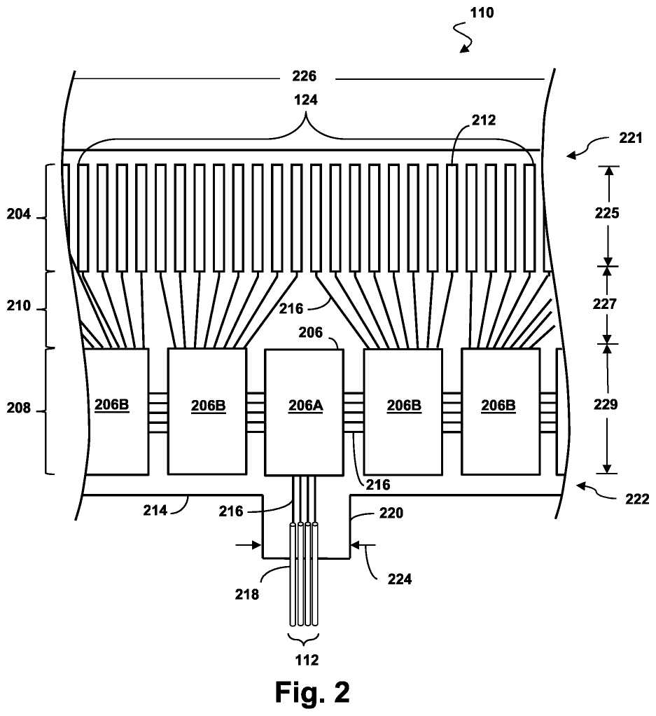

FIG. 2 is a diagrammatic top view of a portion of a flexible assembly 110, according to aspects of the present disclosure. The flexible assembly 110 includes a transducer array 124 formed in a transducer region 204 and transducer control logic dies 206 (including dies 206A and 206B) formed in a control region 208, with a transition region 210 disposed therebetween. The transducer array 124 includes an array of ultrasound transducer elements 212. The transducer control logic dies 206 are mounted on a flexible substrate 214 into which the transducer elements 212 have been previously integrated. The flexible substrate 214 is shown in a flat configuration in FIG. 2. Though six control logic dies 206 are shown in FIG. 2, any number of control logic dies 206 may be used. For example, one, two, three, four, five, six, seven, eight, nine, ten, or more control logic dies 206 may be used.

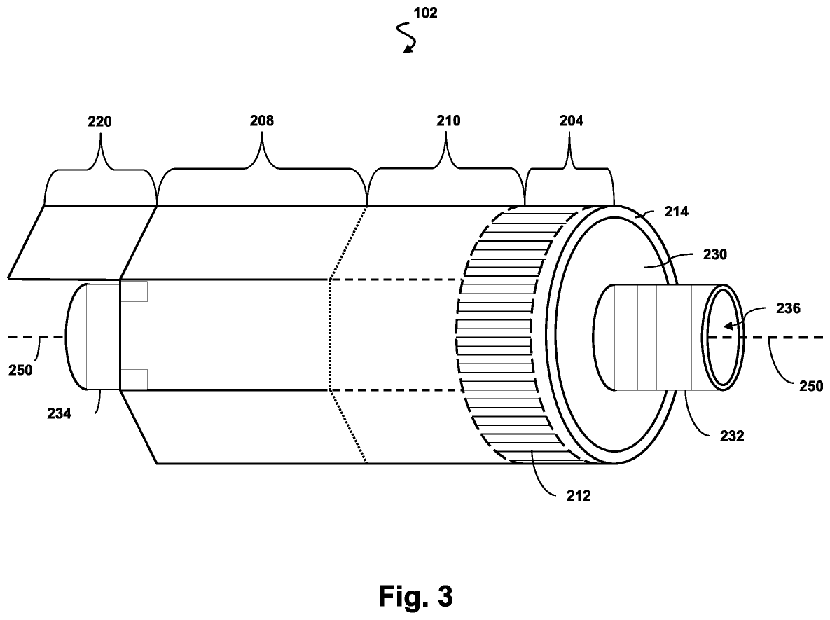

The flexible substrate 214, on which the transducer control logic dies 206 and the transducer elements 212 are mounted, provides structural support and interconnects for electrical coupling. The flexible substrate 214 may be constructed to include a film layer of a flexible polyimide material such as KAPTON™ (trademark of DuPont). Other suitable materials include polyester films, polyimide films, polyethylene napthalate films, or polyetherimide films, liquid crystal polymer, other flexible printed semiconductor substrates as well as products such as Upilex® (registered trademark of Ube Industries) and TEFLON® (registered trademark of E.I. du Pont). In the flat configuration illustrated in FIG. 2, the flexible substrate 214 has a generally rectangular shape. As shown and described herein, the flexible substrate 214 is configured to be wrapped around a support member 230 (FIG. 3) in some instances. Therefore, the thickness of the film layer of the flexible substrate 214 is generally related to the degree of curvature in the final assembled flexible assembly 110. In some embodiments, the film layer is between 5 μm and 100 μm, with some particular embodiments being between 5 μm and 25.1 μm, e.g., 6 μm.

The set of transducer control logic dies 206 is a non-limiting example of a control circuit. The transducer region 204 is disposed at a distal portion 221 of the flexible substrate 214. The control region 208 is disposed at a proximal portion 222 of the flexible substrate 214. The transition region 210 is disposed between the control region 208 and the transducer region 204. Dimensions of the transducer region 204, the control region 208, and the transition region 210 (e.g., lengths 225, 227, 229) can vary in different embodiments. In some embodiments, the lengths 225, 227, 229 can be substantially similar or, the length 227 of the transition region 210 may be less than lengths 225 and 229, the length 227 of the transition region 210 can be greater than lengths 225, 229 of the transducer region and controller region, respectively.

The control logic dies 206 are not necessarily homogenous. In some embodiments, a single controller is designated a master control logic die 206A and contains the communication interface for cable 112, between a processing system, e.g., processing system 106, and the flexible assembly 110. Accordingly, the master control circuit may include control logic that decodes control signals received over the cable 112, transmits control responses over the cable 112, amplifies echo signals, and/or transmits the echo signals over the cable 112. The remaining controllers are slave controllers 206B. The slave controllers 206B may include control logic that drives a plurality of transducer elements 512 positioned on a transducer element 212 to emit an ultrasonic signal and selects a transducer element 212 to receive an echo. In the depicted embodiment, the master controller 206A does not directly control any transducer elements 212. In other embodiments, the master controller 206A drives the same number of transducer elements 212 as the slave controllers 206B or drives a reduced set of transducer elements 212 as compared to the slave controllers 206B. In an exemplary embodiment, a single master controller 206A and eight slave controllers 206B are provided with eight transducers assigned to each slave controller 206B.

To electrically interconnect the control logic dies 206 and the transducer elements 212, in an embodiment, the flexible substrate 214 includes conductive traces 216 formed in the film layer that carry signals between the control logic dies 206 and the transducer elements 212. In particular, the conductive traces 216 providing communication between the control logic dies 206 and the transducer elements 212 extend along the flexible substrate 214 within the transition region 210. In some instances, the conductive traces 216 can also facilitate electrical communication between the master controller 206A and the slave controllers 206B. The conductive traces 216 can also provide a set of conductive pads that contact the conductors 218 of cable 112 when the conductors 218 of the cable 112 are mechanically and electrically coupled to the flexible substrate 214. Suitable materials for the conductive traces 216 include copper, gold, aluminum, silver, tantalum, nickel, and tin, and may be deposited on the flexible substrate 214 by processes such as sputtering, plating, and etching. In an embodiment, the flexible substrate 214 includes a chromium adhesion layer. The width and thickness of the conductive traces 216 are selected to provide proper conductivity and resilience when the flexible substrate 214 is rolled. In that regard, an exemplary range for the thickness of a conductive trace 216 and/or conductive pad is between 1-5 μm. For example, in an embodiment, 5 μm conductive traces 216 are separated by 5 μm of space. The width of a conductive trace 216 on the flexible substrate may be further determined by the width of the conductor 218 to be coupled to the trace or pad.

The flexible substrate 214 can include a conductor interface 220 in some embodiments. The conductor interface 220 can be in a location of the flexible substrate 214 where the conductors 218 of the cable 112 are coupled to the flexible substrate 214. For example, the bare conductors of the cable 112 are electrically coupled to the flexible substrate 214 at the conductor interface 220. The conductor interface 220 can be tab extending from the main body of flexible substrate 214. In that regard, the main body of the flexible substrate 214 can refer collectively to the transducer region 204, controller region 208, and the transition region 210. In the illustrated embodiment, the conductor interface 220 extends from the proximal portion 222 of the flexible substrate 214. In other embodiments, the conductor interface 220 is positioned at other parts of the flexible substrate 214, such as the distal portion 221, or the flexible substrate 214 may lack the conductor interface 220. A value of a dimension of the tab or conductor interface 220, such as a width 224, can be less than the value of a dimension of the main body of the flexible substrate 214, such as a width 226. In some embodiments, the substrate forming the conductor interface 220 is made of the same material(s) and/or is similarly flexible as the flexible substrate 214. In other embodiments, the conductor interface 220 is made of different materials and/or is comparatively more rigid than the flexible substrate 214. For example, the conductor interface 220 can be made of a plastic, thermoplastic, polymer, hard polymer, etc., including polyoxymethylene (e.g., DELRIN®), polyether ether ketone (PEEK), nylon, Liquid Crystal Polymer (LCP), and/or other suitable materials.

FIG. 3 illustrates a perspective view of the scanner assembly 110 in a rolled configuration. In some instances, the flexible substrate 214 is transitioned from a flat configuration (FIG. 2) to a rolled or more cylindrical configuration (FIG. 3). For example, in some embodiments, techniques are utilized as disclosed in one or more of U.S. Pat. No. 6,776,763, titled “ULTRASONIC TRANSDUCER ARRAY AND METHOD OF MANUFACTURING THE SAME” and U.S. Pat. No. 7,226,417, titled “HIGH RESOLUTION INTRAVASCULAR ULTRASOUND SENSING ASSEMBLY HAVING A FLEXIBLE SUBSTRATE,” each of which is hereby incorporated by reference in its entirety.

Depending on the application and embodiment of the presently disclosed invention, transducer elements 212 may be piezoelectric transducers, single crystal transducer, or PZT (lead zirconate titanate) transducers. In other embodiments, the transducer elements of transducer array 124 may be flexural transducers, piezoelectric micromachined ultrasonic transducers (PMUTs), capacitive micromachined ultrasonic transducers (CMUTs), or any other suitable type of transducer element. In such embodiments, transducer elements 212 may comprise an elongate semiconductor material or other suitable material that allows micromachining or similar methods of disposing extremely small elements or circuitry on a substrate.

In some embodiments, the transducer elements 212 and the controllers 206 can be positioned in an annular configuration, such as a circular configuration or in a polygon configuration, around a longitudinal axis 250 of a support member 230. It is understood that the longitudinal axis 250 of the support member 230 may also be referred to as the longitudinal axis of the scanner assembly 110, the flexible elongate member 121, or the device 102. For example, a cross-sectional profile of the imaging assembly 110 at the transducer elements 212 and/or the controllers 206 can be a circle or a polygon. Any suitable annular polygon shape can be implemented, such as one based on the number of controllers or transducers, flexibility of the controllers or transducers, etc. Some examples may include a pentagon, hexagon, heptagon, octagon, nonagon, decagon, etc. In some examples, the transducer controllers 206 may be used for controlling the ultrasound transducers 512 of transducer elements 212 to obtain imaging data associated with the vessel 120.

The support member 230 can be referenced as a unibody in some instances. The support member 230 can be composed of a metallic material, such as stainless steel, or a non-metallic material, such as a plastic or polymer as described in U.S. Provisional Application No. 61/985,220, “Pre-Doped Solid Substrate for Intravascular Devices,” filed Apr. 28, 2014, the entirety of which is hereby incorporated by reference herein. In some embodiments, support member 230 may be composed of 303 stainless steel. The support member 230 can be a ferrule having a distal flange or portion 232 and a proximal flange or portion 234. The support member 230 can be tubular in shape and define a lumen 236 extending longitudinally therethrough. The lumen 236 can be sized and shaped to receive the guide wire 118. The support member 230 can be manufactured using any suitable process. For example, the support member 230 can be machined and/or electrochemically machined or laser milled, such as by removing material from a blank to shape the support member 230, or molded, such as by an injection molding process or a micro injection molding process.

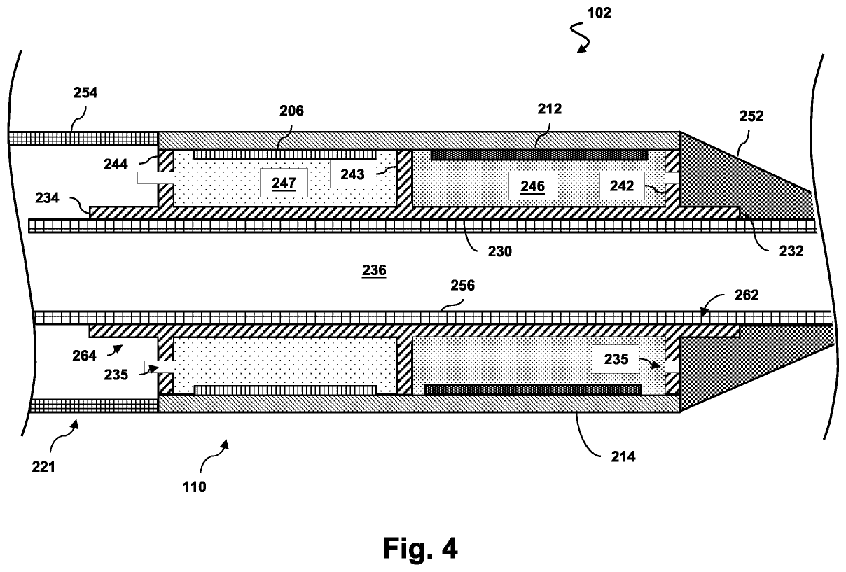

Referring now to FIG. 4, shown therein is a diagrammatic cross-sectional side view of a distal portion of the intraluminal imaging device 102, including the flexible substrate 214 and the support member 230, according to aspects of the present disclosure. The lumen 236 may be connected with the entry/exit port 116 and is sized and shaped to receive the guide wire 118 (FIG. 1). In some embodiments, the support member 230 may be integrally formed as a unitary structure, while in other embodiments the support member 230 may be formed of different components, such as a ferrule and stands 242, 243, and 244, that are fixedly coupled to one another. In some cases, the support member 230 and/or one or more components thereof may be completely integrated with inner member 256. In some cases, the inner member 256 and the support member 230 may be joined as one, e.g., in the case of a polymer support member.

Stands 242, 243, and 244 that extend vertically are provided at the distal, central, and proximal portions respectively, of the support member 230. The stands 242, 243, and 244 elevate and support the distal, central, and proximal portions of the flexible substrate 214. In that regard, portions of the flexible substrate 214, such as the transducer portion 204 (or transducer region 204), can be spaced from a central body portion of the support member 230 extending between the stands 242, 243, and 244. The stands 242, 243, 244 can have the same outer diameter or different outer diameters. For example, the distal stand 242 can have a larger or smaller outer diameter than the central stand 243 and/or proximal stand 244 and can also have special features for rotational alignment as well as control chip placement and connection.

To improve acoustic performance, the cavity between the transducer array 212 and the surface of the support member 230 may be filled with an acoustic backing material 246. The liquid backing material 246 can be introduced between the flexible substrate 214 and the support member 230 via passageway 235 in the stand 242, or through additional recesses as will be discussed in more detail hereafter. The backing material 246 may serve to attenuate ultrasound energy emitted by the transducer array 212 that propagates in the undesired, inward direction.

The cavity between the circuit controller chips 206 and the surface of the support member 230 may be filled with an underfill material 247. The underfill material 247 may be an adhesive material (e.g. an epoxy) which provides structural support for the circuit controller chips 206 and/or the flexible substrate 214. The underfill 247 may additionally be any suitable material.

In some embodiments, the central body portion of the support member can include recesses allowing fluid communication between the lumen of the unibody and the cavities between the flexible substrate 214 and the support member 230. Acoustic backing material 246 and/or underfill material 247 can be introduced via the cavities (during an assembly process, prior to the inner member 256 extending through the lumen of the unibody. In some embodiments, suction can be applied via the passageways 235 of one of the stands 242, 244, or to any other suitable recess while the liquid backing material 246 is fed between the flexible substrate 214 and the support member 230 via the passageways 235 of the other of the stands 242, 244, or any other suitable recess. The backing material can be cured to allow it to solidify and set. In various embodiments, the support member 230 includes more than three stands 242, 243, and 244, only one or two of the stands 242, 243, 244, or none of the stands. In that regard the support member 230 can have an increased diameter distal portion 262 and/or increased diameter proximal portion 264 that is sized and shaped to elevate and support the distal and/or proximal portions of the flexible substrate 214.

The support member 230 can be substantially cylindrical in some embodiments. Other shapes of the support member 230 are also contemplated including geometrical, non-geometrical, symmetrical, non-symmetrical, cross-sectional profiles. As the term is used herein, the shape of the support member 230 may reference a cross-sectional profile of the support member 230. Different portions of the support member 230 can be variously shaped in other embodiments. For example, the proximal portion 264 can have a larger outer diameter than the outer diameters of the distal portion 262 or a central portion extending between the distal and proximal portions 262, 264. In some embodiments, an inner diameter of the support member 230 (e.g., the diameter of the lumen 236) can correspondingly increase or decrease as the outer diameter changes. In other embodiments, the inner diameter of the support member 230 remains the same despite variations in the outer diameter.

A proximal inner member 256 and a proximal outer member 254 are coupled to the proximal portion 264 of the support member 230. The proximal inner member 256 and/or the proximal outer member 254 can comprise a flexible elongate member. The proximal inner member 256 can be received within a proximal flange 234. The proximal outer member 254 abuts and is in contact with the proximal end of flexible substrate 214. A distal tip member 252 is coupled to the distal portion 262 of the support member 230. For example, the distal member 252 is positioned around the distal flange 232. The tip member 252 can abut and be in contact with the distal end of flexible substrate 214 and the stand 242. In other embodiments, the proximal end of the tip member 252 may be received within the distal end of the flexible substrate 214 in its rolled configuration. In some embodiments there may be a gap between the flexible substrate 214 and the tip member 252. The distal member 252 can be the distal-most component of the intraluminal imaging device 102. The distal tip member 252 may be a flexible, polymeric component that defines the distal-most end of the imaging device 102. The distal tip member 252 may additionally define a lumen in communication with the lumen 236 defined by support member 230. The guide wire 118 may extend through lumen 236 as well as the lumen defined by the tip member 252.

One or more adhesives can be disposed between various components at the distal portion of the intraluminal imaging device 102. For example, one or more of the flexible substrate 214, the support member 230, the distal member 252, the proximal inner member 256, the transducer array 212, and/or the proximal outer member 254 can be coupled to one another via an adhesive. Stated differently, the adhesive can be in contact with e.g. the transducer array 212, the flexible substrate 214, the support member 230, the distal member 252, the proximal inner member 256, and/or the proximal outer member 254, among other components.

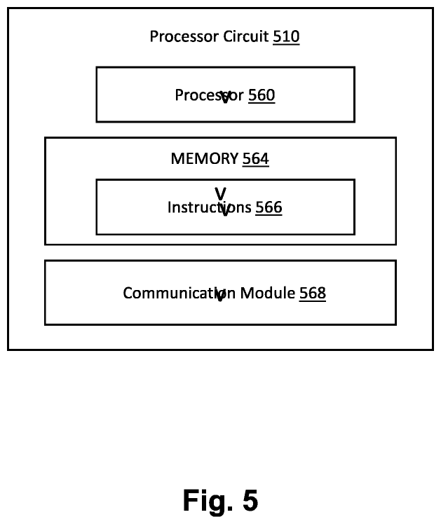

FIG. 5 is a schematic diagram of a processor circuit, according to aspects of the present disclosure. The processor circuit 510 may be implemented in the control system 130 of FIG. 1, the intraluminal imaging system 101, and/or the x-ray imaging system 151, or any other suitable location. In an example, the processor circuit 510 may be in communication with intraluminal imaging device 102, the x-ray imaging device 152, the display 132 within the system 100. The processor circuit 510 may include the processor 134 and/or the communication interface 140 (FIG. 1). One or more processor circuits 510 are configured to execute the operations described herein. As shown, the processor circuit 510 may include a processor 560, a memory 564, and a communication module 568. These elements may be in direct or indirect communication with each other, for example via one or more buses.

The processor 560 may include a CPU, a GPU, a DSP, an application-specific integrated circuit (ASIC), a controller, an FPGA, another hardware device, a firmware device, or any combination thereof configured to perform the operations described herein. The processor 560 may also be implemented as a combination of computing devices, e.g., a combination of a DSP and a microprocessor, a plurality of microprocessors, one or more microprocessors in conjunction with a DSP core, or any other such configuration.

The memory 564 may include a cache memory (e.g., a cache memory of the processor 560), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 564 includes a non-transitory computer-readable medium. The memory 564 may store instructions 566. The instructions 566 may include instructions that, when executed by the processor 560, cause the processor 560 to perform the operations described herein with reference to the probe 110 and/or the host 130 (FIG. 1). Instructions 566 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 568 can include any electronic circuitry and/or logic circuitry to facilitate direct or indirect communication of data between the processor circuit 510, the probe 110, and/or the display 132 and/or display 132. In that regard, the communication module 568 can be an input/output (I/O) device. In some instances, the communication module 568 facilitates direct or indirect communication between various elements of the processor circuit 510 and/or the probe 110 (FIG. 1) and/or the host 130 (FIG. 1).

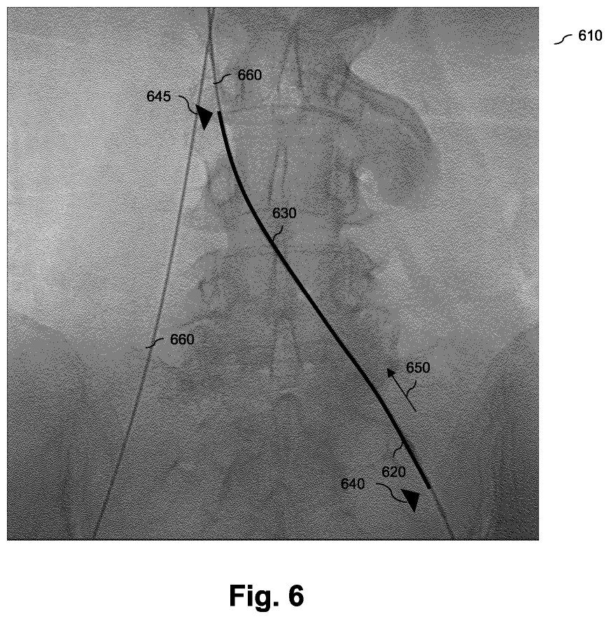

FIG. 6 is a diagrammatic view of an x-ray fluoroscopy image illustrating a pullback procedure, according to aspects of the present disclosure. FIG. 6 depicts an x-ray fluoroscopy image 610 showing an intravascular device 620 and guidewires 660. FIG. 6 additionally depicts an intravascular device path 630, a starting indicator 640, an ending indicator 645, and a directional arrow 650.

During a pullback procedure, one or more guidewires 660 may be positioned within one or more lumens of a patient. Because the guidewire 660 may be constructed of a flexible material, the shape of the guidewire 660 may conform to the shape of the lumen in which the guidewire 660 is positioned. The guidewire 660 may include a flexible elongate member. An intravascular device 620 may be positioned within the lumen and travel through the lumen along a guidewire 660, which is positioned within a guidewire lumen of the intravascular device 620. The intravascular device 620 can be a catheter or a guide catheter. The intravascular device 620 may be an IVUS catheter. The device 620 may be constructed of a flexible material, such that the shape of the device 620 may match the curvature of the lumen in which the device 620 is positioned. The intravascular device 620 may include a flexible elongate member. In the fluoroscopy image 610, a radiopaque portion of the intravascular device 620 is visible. The intravascular device 620 may be substantially similar to the device 102 of the intraluminal ultrasound imaging system 101. A user of the system 100 may position the intravascular device 620 at a starting location shown by the indicator 640. With the intravascular device 620 placed at the starting location, the user may begin acquiring fluoroscopy images with the x-ray imaging system 151. The image 610 may be one of the many x-ray fluoroscopy images obtained during the pullback. In some embodiments, the fluoroscopy image 610 is an x-ray image obtained while no contrast agent is present within the patient anatomy. In such an embodiment, the lumens (e.g., blood vessel) of the patient may be identified primarily by the positioning of the guidewires 660 within the lumens. In other embodiments, the image 610 may be an x-ray image obtained while a contrast agent is present within the patient anatomy. The contrast agent may make vessel lumens visible within the image 610.

One or a plurality of radiopaque portions of the guidewire 660 are visible in the x-ray image(s) 610 obtained with or without contrast. The radiopaque portions can be one length or a plurality of lengths of the guidewire 660. In some embodiments, the radiopaque portions of the guidewire 660 are one or a plurality of radiopaque markers. The radiopaque markers can be made of a different material that is more radiopaque than the material used to form other parts of the guidewire 660. In some embodiments, all or substantially all of the guidewire 660 can be radiopaque. In some embodiments, all or substantially all of the portion of the guidewire 660 within the patient body can be radiopaque. In some embodiments, all or substantially all of the distal portion of the guidewire 660 (e.g., the portion of the guidewire being imaged by x-ray) can be radiopaque. For example, the guidewire 660 can be sufficiently thick (e.g., a sufficiently large diameter) to provide radiopacity in x-ray images 610. Such embodiments can include clinical applications in the peripheral venous system, which can involve guidewires with a diameter between 0.014″ and 0.038″, including values such as 0.014″, 0.018″, 0.035″, 0.038″, and/or other values both larger and smaller.

While the x-ray imaging system 151 acquires fluoroscopy images, the user of the system 100 may then begin to move device 620 through the patient lumen along the guidewire 660. The user may pull the device in a direction shown by the arrow 650. As the device 620 moves along the guidewire 660 through the lumen, the device 620 shown in newly acquired fluoroscopy images is shown to move in the direction shown by the arrow 650. The user may continue to pull the device 620 along the guidewire 660 until an ending position 645. The path taken by the device 620 during the pullback procedure may be illustrated by the path 630 within FIG. 6.

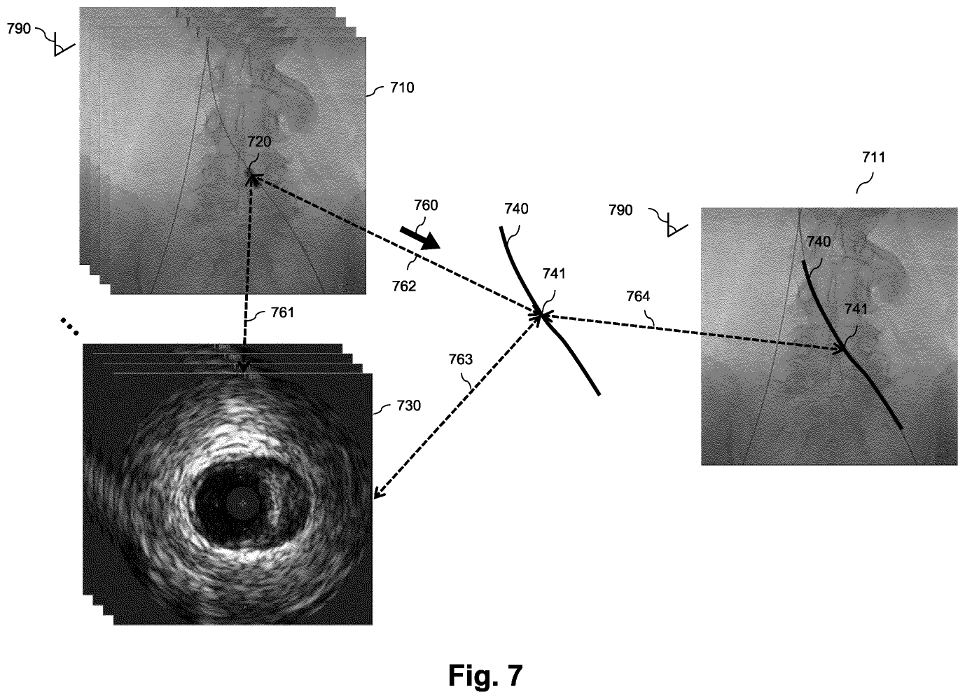

As the device 620 moves from the starting position shown by the indicator 640 to the ending position shown by the indicator 645, it may acquire any suitable intravascular data, such as IVUS images. After the device 620 has moved to the ending position, the user may stop acquiring fluoroscopy images with the x-ray imaging system 151 and may remove the device 620 from the lumen. Because the intravascular data was obtained with the device 620 while fluoroscopy images were simultaneously acquired, the intravascular data may be coregistered to the places along the path 630 at which each datum was collected and displayed in relation to that location along the path 630 and/or a representative fluoroscopy image as will be described with greater detail with reference to FIG. 7.

In some embodiments, the intravascular device 620 may be moved in an opposite direction. For example, the device may be moved from the position of indicator 645 to the position of indicator 640. In other words, the device 620 may move from a distal region to a proximal region (e.g., a pullback) or may move from a proximal region to a distal region (e.g., push forward) during the imaging procedure.

It is noted, that the starting and ending positions may represent target locations during an IVUS imaging procedure. Any indicators, such as indicators 640 and/or 645, identifying these locations may not be visible within an x-ray image displayed to a user during a pullback procedure. For example, during an imaging procedure, the system may identify the starting location of the device 620 on the display, but the ending location of the device 620 is not known because the procedure is still in the process of being completed. However, after an IVUS imaging procedure or pullback procedure is completed, during a review phase of the process, indicators 640 and/or 645 identifying both the starting location and the ending location may be displayed to a user of the system.

FIG. 7 is a diagrammatic view of a relationship between x-ray fluoroscopy images 710, intravascular data 730, and a path 740 defined by the motion of an intravascular device, according to aspects of the present disclosure. FIG. 7 describes a method of coregistering intravascular data 730 including intravascular images with corresponding locations on one or more fluoroscopy images 710 of the same region of a patient's anatomy.

The patient anatomy may be imaged with an x-ray device while a physician performs a pullback with an intravascular device 720, e.g., while the intravascular device 720 moves through a blood vessel of the anatomy. The intravascular device may be substantially similar to the intravascular device 102 described with reference to FIG. 1. The x-ray device used to obtain the fluoroscopy images 710 may be substantially similar to the x-ray device 152 of FIG. 1. In some embodiments, the fluoroscopy images 710 may be obtained while no contrast agent is present within the patient vasculature. Such an embodiment is shown by the fluoroscopy images 710 in FIG. 7. The radiopaque portion of the intravascular device 720 is visible within the fluoroscopy image 710. The fluoroscopy images 710 may correspond to a continuous image stream of fluoroscopy images and may be obtained as the patient anatomy is exposed to a reduced dose of x-radiation. It is noted that the fluoroscopy images 710 may be acquired with the x-ray source 160 and the x-ray detector 170 positioned at any suitable angle in relation to the patient anatomy. This angle is shown by angle 790.

The intravascular device 720 may be any suitable intravascular device. As the intravascular device 720 moves through the patient vasculature, the x-ray imaging system may acquire multiple fluoroscopy images 710 showing the radiopaque portion of the intravascular device 720. In this way, each fluoroscopy image 710 shown in FIG. 7 may depict the intravascular device 720 positioned at a different location such that a processor circuit may track the position of the intravascular device 720 over time.

As the intravascular device 720 is pulled through the patient vasculature, it may acquire intravascular data 730. In an example, the intravascular data 730 shown in FIG. 7 may be IVUS images. However, the intravascular data may be any suitable data, including IVUS images, FFR data, iFR data, OCT images, intravascular photoacoustic (IVPA) images, or any other measurements or metrics relating to blood pressure, blood flow, lumen structure, or other physiological data acquired during a pullback of an intravascular device.

As the physician pulls the intravascular device 720 through the patient vasculature, each intravascular data point 730 acquired by the intravascular device 720 may be associated with a position within the patient anatomy in the fluoroscopy images 710, as indicated by the arrow 761. For example, the first IVUS image 730 shown in FIG. 7 may be associated with the first fluoroscopy image 710. The first IVUS image 730 may be an image acquired by the intravascular device 720 at a position within the vasculature, as depicted in the first fluoroscopy image 710 as shown by the intravascular device 720 within the image 710. Similarly, an additional IVUS image 730 may be associated with an additional fluoroscopy image 710 showing the intravascular device 720 at a new location within the image 710, and so on. The processor circuit may determine the locations of the intravascular device 720 within each acquired x-ray image 710 by any suitable method. For example, the processor circuit may perform various image processing techniques, such as edge identification of the radiopaque marker, pixel-by-pixel analysis to determine transition between light pixels and dark pixels, filtering, or any other suitable techniques to determine the location of the imaging device 720. In some embodiments, the processor circuit may use various artificial intelligence methods including deep learning techniques such as neural networks or any other suitable techniques to identify the locations of the imaging device 720 within the x-ray images 710.

Any suitable number of IVUS images or other intravascular data points 730 may be acquired during an intravascular device pullback and any suitable number of fluoroscopy images 710 may be obtained. In some embodiments, there may be a one-to-one ratio of fluoroscopy images 710 and intravascular data 730. In other embodiments, there may be differing numbers of fluoroscopy images 710 and/or intravascular data 730. The process of co-registering the intravascular data 730 with one or more x-ray images may include some features similar to those described in U.S. Pat. No. 7,930,014, titled, “VASCULAR IMAGE CO-REGISTRATION,” and filed Jan. 11, 2006, which is hereby incorporated by reference in its entirety. The co-registration process may also include some features similar to those described in U.S. Pat. Nos. 8,290,228, 8,463,007, 8,670,603, 8,693,756, 8,781,193, 8,855,744, and 10,076,301, all of which are also hereby incorporated by reference in their entirety.

The system 100 may additionally generate a fluoroscopy-based 2D pathway 740 defined by the positions of the intravascular device 720 within the x-ray fluoroscopy images 710. The different positions of the intravascular device 720 during pullback, as shown in the fluoroscopy images 710, may define a two-dimensional pathway 740, as shown by the arrow 760. The fluoroscopy-based 2D pathway 740 reflects the path of one or more radiopaque portions of the intravascular device 720 as it moved through the patient vasculature as observed from the angle 790 by the x-ray imaging device 152. The fluoroscopy-based 2D pathway 740 defines the path as measured by the x-ray device which acquired the fluoroscopy images 710, and therefore shows the path from the same angle 790 at which the fluoroscopy images were acquired. Stated differently, the 2D pathway 740 describes the projection of the 3D path followed by the device onto the imaging plane at the imaging angle 790. In some embodiments, the pathway 740 may be determined by an average of the detected locations of the intravascular device 720 in the fluoroscopy images 710. For example, the pathway 740 may not coincide exactly with the guidewire in any fluoroscopy image 710 selected for presentation.

As shown by the arrow 762, because the two-dimensional path 740 is generated based on the fluoroscopy images 710, each position along the two-dimensional path 740 may be associated with one or more fluoroscopy images 710. As an example, at a location 741 along the path 740, the first fluoroscopy image 710 may depict the intravascular device 720 at that same position 741. In addition, because a correspondence was also established between the fluoroscopy images 710 and the intravascular data 730 as shown by the arrow 761, intravascular data 730, such as the first IVUS image shown, may also be associated with the location 741 along the path 740 as shown by the arrow 763.

Finally, the path 740 generated based on the locations of the intravascular device 720 within the fluoroscopy images 710 may be overlaid onto any suitable fluoroscopy image 711 (e.g., one of the fluoroscopic images 710 in the fluoroscopic image stream). In this way, any location along the path 740 displayed on the fluoroscopy image 711 may be associated with IVUS data such as an IVUS image 730, as shown by the arrow 764. For example, IVUS image 730 shown in FIG. 7 may be acquired simultaneously with the fluoroscopy image 710 shown and the two may be associated with each other as shown by the arrow 761. The fluoroscopy image 710 may then indicate the location of the intravascular device 720 along the path 740, as shown by the arrow 762, thus associating the IVUS image 730 with the location 741 along the path 740 as shown by the arrow 763. Finally, the IVUS image 730 may be associated with the location within the fluoroscopy image 710 at which it was acquired by overlaying the path 740 with associated data on the fluoroscopy image 711. The pathway 740 itself may or may not be displayed on the image 711.

In the illustrated embodiment of FIG. 7, the co-registered IVUS images are associated with one of the fluoroscopic images obtained without contrast such that that the position at which the IVUS images are obtained is known relative to locations along the guidewire. In other embodiments, the co-registered IVUS images are associated with an x-ray image obtained with contrast (in which the vessel is visible) such that the position at which the IVUS images are obtained is known relative to locations along the vessel.

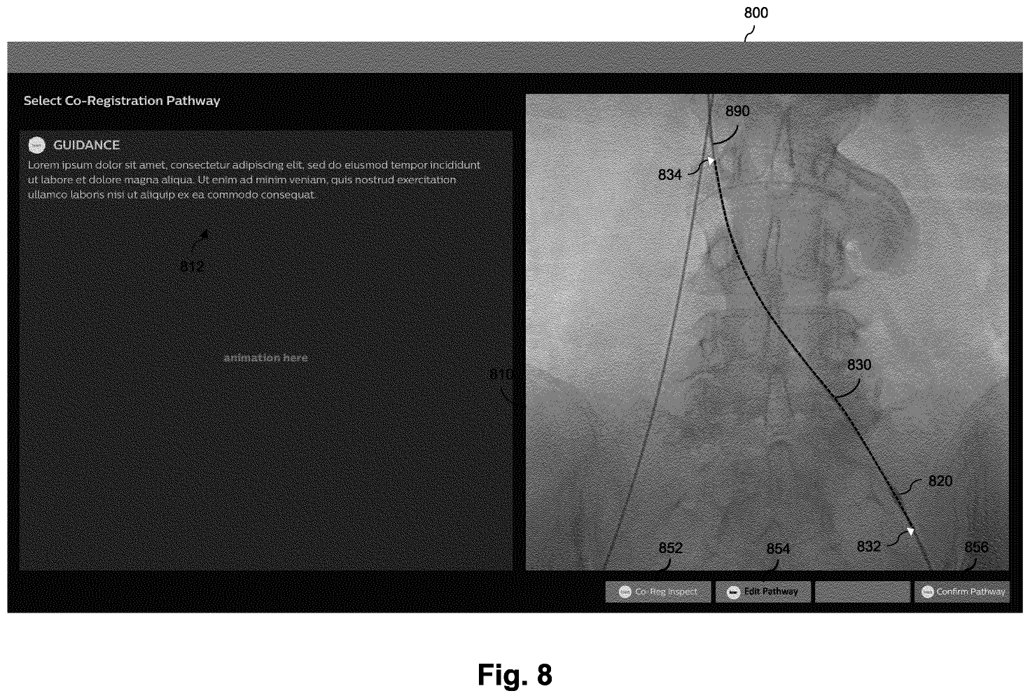

FIG. 8 is a diagrammatic view of a graphical user interface 800 displaying a coregistration pathway 830 overlaid on an x-ray image 810, according to aspects of the present disclosure. FIG. 8 includes an x-ray image 810 showing a depiction of a guidewire 890 because the guidewire 890 is radiopaque. Overlaid over the guidewire 890 is the co-registration pathway 830 with a starting position 832 and an ending position 834. An imaging device 820 is also shown along the guidewire 890 (because it is radiopaque) on the pathway 830 between the starting position 832 and the ending position 834. FIG. 8 also depicts accompanying text 812, and soft buttons 852, 854, and 856.

After an intravascular imaging procedure has been performed using an intravascular imaging device 820 and x-ray imaging device, and a processor circuit has completed co-registration with the intravascular images and the x-ray image, an exemplary x-ray image 810 may be displayed to a user of the system 100. The x-ray image 810 may show a view of the patient anatomy including the pathway 830, which represents the distance, region, or trajectory within the patient vessel that the device 820 travelled.

The guidewire 890 shown within the x-ray image 810 may be used to identify the location of the vessel that was imaged. Because the guidewire has radiopaque properties, it is easily seen on an x-ray image. By inserting the guidewire 890 through a vessel to be imaged, radiopaque contrast is not needed to identify the imaged vessel, which advantageously eliminates a step from the imaging process and making the procedure simpler and faster. In addition, some patients may be sensitive to radiopaque contrast due to various conditions, such as impaired kidney function. Avoiding radiopaque contrast in such situations is clinically advantageous because it avoids risk of harm to the patient. Accordingly, the vessel that was imaged by the intravascular imaging device 820 can be indirectly visualized because the guidewire 890 has the same shape as the vessel when the guidewire 890 is positioned within the vessel. Any number of guidewires may be displayed within the x-ray image 810 (e.g., different guidewires positioned inside different vessels, multiple guidewires positioned inside a given vessel). In some embodiments, the pathway 830 may not be displayed overlaid on the x-ray image 810. In addition, any of the x-ray images acquired during an imaging procedure may be used as the image 810. Any of these images may have the pathway 830 overlaid over them or may not and may be presented to the user in a graphical user interface like the interface 800.

The imaging device 820 may include a lumen 236 extending through the device 820, as explained with reference to FIG. 3. The device 820 may be positioned so that the guidewire 890 passes through this lumen 236. In this configuration, as the user of the system 100 pushes or pulls the catheter 820, the catheter 820 may move in either a distal or proximal direction within the patient vessel as it is guided along the guidewire 890. The imaging device 820 may alternatively be referred to as an imaging assembly, probe, intraluminal imaging device, catheter, or any other suitable term.

Because the imaging device 820 is constructed of radiopaque materials, like the guidewire 890, it may also be seen within the x-ray image 810. For example, the device 820 is shown in FIG. 8 positioned along the guidewire 890. For example, the imaging assembly (including a transducer and/or a transducer array) at the distal portion of an IVUS catheter, and/or one or more radiopaque markers of the device 820 can be visualized in the x-ray image.

As explained with reference to FIG. 6, during IVUS image acquisition, the imaging device may be moved to a predetermined starting position along the vessel to be imaged. The predetermined starting position, as well as a predetermined ending position, may be determined by the user of the system 100 or by a physician before the procedure. In one example, the predetermined starting position may be some position along the guidewire 890 near the indicator 832 shown in FIG. 8. The predetermined ending position may be some position along the guidewire 890 near the indicator 834 shown in FIG. 8. For example, the predetermined starting position may be a location along the guidewire 890 or vessel distal to the predetermined ending position such that the device 820 is moved in a proximal direction throughout the imaging procedure. In some embodiments, however, the predetermined starting position may be proximal to the predetermined ending position, such that the device moves in a distal direction throughout the procedure. The predetermined starting position and predetermined ending position may be determined based on symptoms or conditions of the patient or based on regions of interest already identified within the patient. After the imaging device 820 is positioned near the predetermined starting location, the user may ensure that both the device 820 is acquiring intraluminal data and the x-ray imaging device 152 is acquiring x-ray images. The user may then move the device 820 from the predetermined starting position along the guidewire 890 to the predetermined ending position. The movement of the device 820 from the predetermined starting position to the predetermined ending position may define the path 830. The device 820 may acquire intraluminal data at all locations along this path 830. The guidewire 890 may be stationary as the imaging catheter 820 moves along the guidewire 890. In some embodiments, the guidewire 890 may shift during a pullback procedure but retain the same general profile or shape as observed in the x-ray image 810. For example, the guidewire 890 may shift in a transverse direction, but the guidewire 890 may remain stationary in a longitudinal direction.

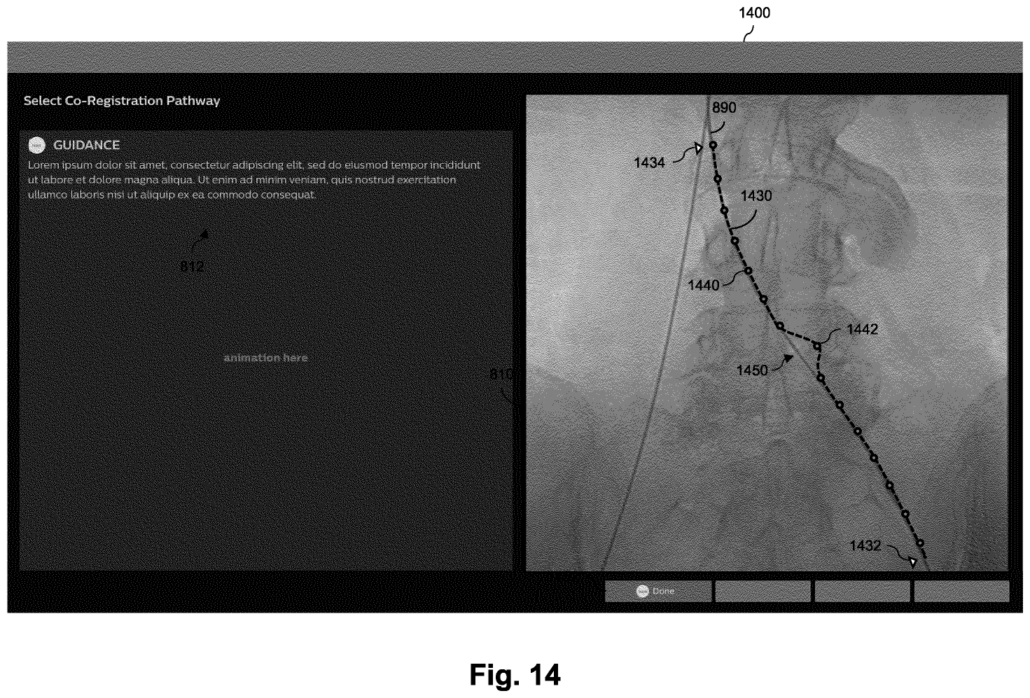

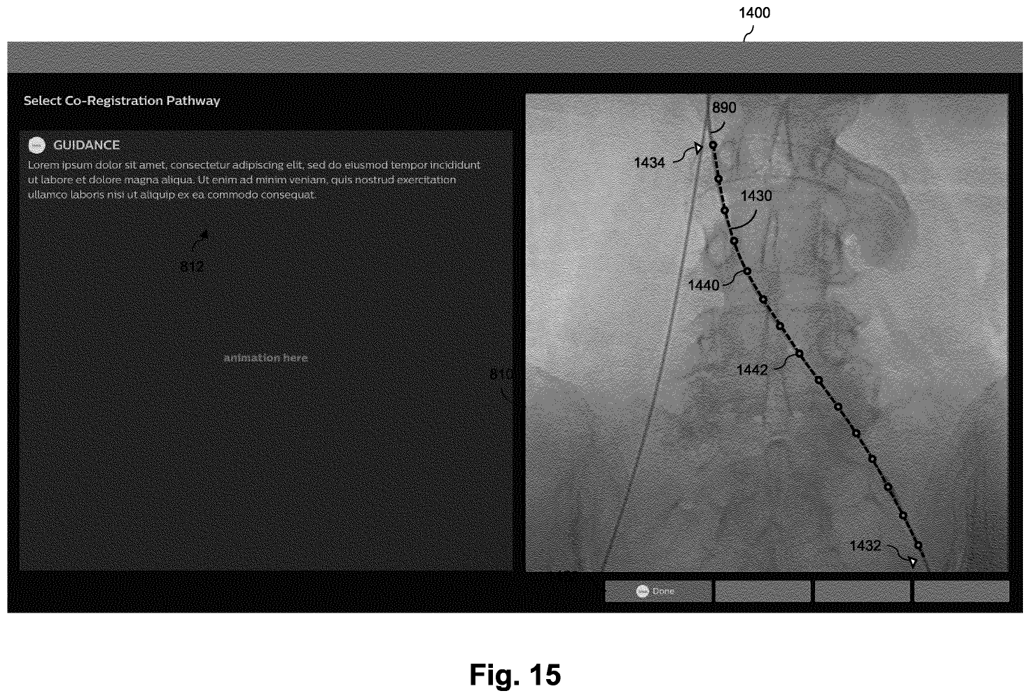

After the IVUS images and x-ray images during IVUS image acquisition are obtained, the processor circuit completes co-registration. During co-registration, the processor circuit determines the path 830, which represent the locations that the device 820 moved along the guidewire in the x-ray image 810. The x-ray image 810 may be one of the x-ray images obtained during the IVUS imaging procedure. In some embodiments, the x-ray image 810 may represent an x-ray image obtained with the same imaging conditions, including the position of the patient, the imaging angle of the x-ray imaging device, etc. In some embodiments, the x-ray image 810 may have been obtained with or without contrast. The processor circuit may additionally determine the location along the path 830 at which the first IVUS image was obtained by the device 820. The processor circuit may generate an indicator 832 representative of this location of the first acquired IVUS image. Similarly, the processor circuit may determine the location at which the final IVUS image was acquired during the imaging procedure. The processor circuit may generate an indicator 834 representative of this location as well. The indicators 832 and 834 may be displayed or may not be. In some embodiments, the location of the indicator 832 representative of the location of the first acquired IVUS image may correspond to the most distal location of the path 830 as shown in FIG. 8 or may correspond to any other location along the path 830. Similarly, the indicator 834 may identify the most proximal location of the path 830 as shown in FIG. 8 or may correspond to any other location along the path 830. The system may display the path 830 overlaid over the x-ray image 810. As will be discussed with reference to FIG. 14 and FIG. 15, the pathway 830 may align with the guidewire 890 or not align with the guidewire 890 at some locations along the pathway 830.

The graphical user interface 800 may additionally display to the user various controls, including but not limited to the button 852, the button 854, and the button 856. The buttons 852, 854, and 856 are soft buttons that are UI elements in the screen display. The buttons 852, 854, 856 may be actuatable by a mouse click or a user touch on a touchscreen, for example. The buttons 852, 854, 856 may be used by the user to verify that the coregistration of the intraluminal data acquired with the device 820 with locations along the pathway 830. Upon the selection by a user of one or more of the buttons 852, 854, and 856, the processor circuit may initiate any suitable function or feature. For example, the processor circuit may alter the appearance of the graphical user interface 800, initiate any various calculations or metrics relating to acquired or displayed data, or perform any other function. The processor circuit may additionally display any other suitable buttons or controls within the graphical user interface 800.

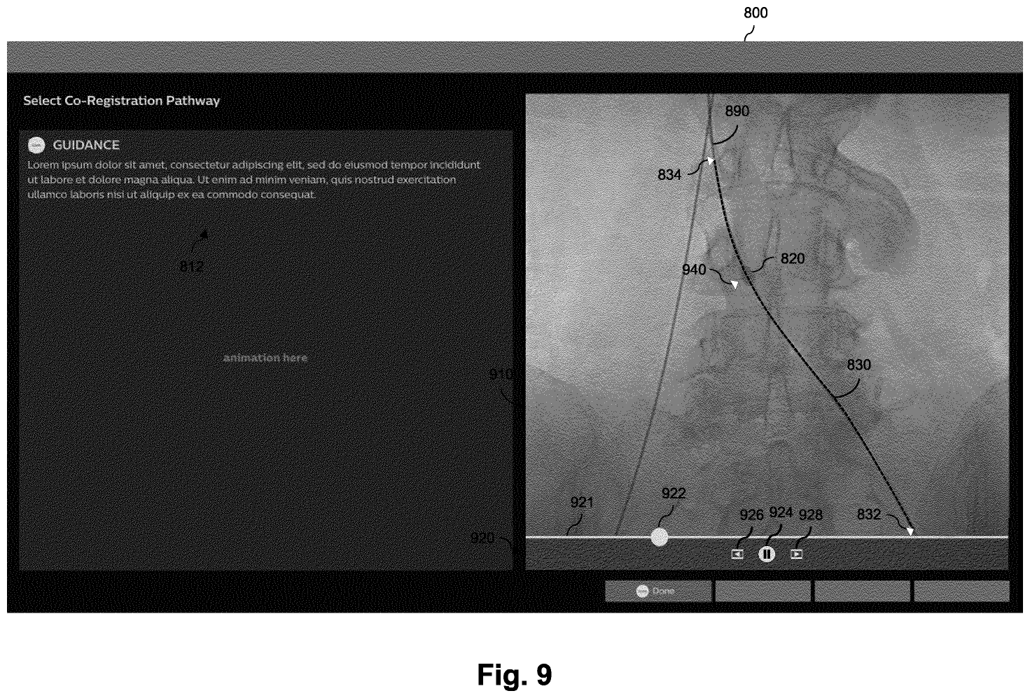

As an example, after receiving a user input selecting the button 852, the processor circuit may display a graphical user interface allowing a user to inspect the accuracy of the coregistration of the intravascular data received by the imaging device 820 with locations within the x-ray image 810. This displayed interface may allow the user to view the x-ray images acquired with the x-ray imaging system 151 to verify that coregistered intravascular data is assigned to correct locations within all the acquired x-ray images. This process will be discussed with more detail with reference to FIGS. 9-13. After receiving a user input selecting the button 854, the processor circuit may display a graphical user interface allowing the user to adjust the shape of the pathway 830 to better align with the shape of the guidewire 890, as will be discussed with reference to FIGS. 14-15. After receiving a user input selecting the button 856, the processor circuit revert to a previously displayed interface after all changes are made to locations of coregistered intravascular data within x-ray images or after the pathway 830 is edited. In some embodiments, a selection of the button 856 may direct the processor circuit of the system 100 to perform or re-perform coregistration of all intravascular data acquired with all corresponding x-ray images to ensure maximum accuracy as will be discussed in more detail hereafter.

The text 812 shown in the graphical user interface 800 may contain any suitable text or information. For example, the text 812 may describe various features of the image 810, the pathway 830, the buttons 852, 854, or 856, or any other features of the interface 800 displayed. The text 812 may include instructions to the user of how to use various features within the interface 800. It may include symbols, pictures, or animations, or any other visual representation.

FIG. 9 is a diagrammatic view of a graphical user interface 800 displaying a marker 940 identifying a location of the imaging device 820 within an x-ray image 910, according to aspects of the present disclosure. The graphical user interface 800 shown in FIG. 9 includes many of the same features described with reference to FIG. 8. The interface 800 in FIG. 9 includes an x-ray image 910, a marker 940 and an x-ray image sequence control panel 920.

In some embodiments, the graphical user interface 800 shown in FIG. 9 may be displayed to a user after the user selects the button 852 (FIG. 8). The user may select the button 852 to verify that the locations where the processor circuit determined the imaging device 820 was positioned when the intraluminal data was acquired by the imaging device 820 matches the locations where the imaging device 820 actually appeared in the x-ray images. If the processor circuit performed imaging processing of the x-ray images correctly, then all of the computed locations should match the actual locations where the radiopaque portion of the imaging device 820 appears in the x-ray images. The system 100 allows the user to perform a manual check of the processor circuit's location computation by pressing the button 852. The system 100 may accomplish this step in any number of suitable ways. In some instances, the interface 800 may display to the user the panel 920.

Like the x-ray image 810 of FIG. 8, the x-ray image 910 may be an image acquired by the x-ray imaging system 151 during the IVUS imaging procedure. For example, images 810 and 910 may be x-ray image frames acquired by the x-ray imaging device during IVUS image acquisition. The image 910 may differ from the image 810 in that the image 910 may have been acquired at a different time during the imaging procedure than the image 810. The image 810 may be different than the image 910 in that the location of radiopaque portion of the imaging device 820 is different. However, in some embodiments, the image 910 may be the same image as the image 810 (e.g., the radiopaque portion of the imaging device 820 may be in the same place).

The processor circuit may display the control panel 920 overlaid over the x-ray image 910 as shown in FIG. 9. The control panel 920 may be positioned near the bottom of the image 910, as shown, or at the top or sides of the image 910, or at any other suitable location. The panel 920 may also be shown adjacent to the image 910 or at any other suitable location within the interface 800.

The panel 920 includes several additional controls. These additional controls may include a timeline 921, a time marker 922, a button 924, and buttons 926 and 928. The timeline 921 may be straight line extending from one side of the x-ray image 810 to the other, or from one side of the panel 920 to the other. This line may represent the time period over which x-ray images were obtained by the system 100 during the IVUS imaging procedure. For example, the location along the timeline 921 at the left side of the line may correspond to the first x-ray image obtained during the IVUS imaging procedure. This first x-ray image may depict the starting location of the IVUS imaging device 820 during the IVUS imaging procedure. Similarly, the location along the timeline 921 at the right side of the line may correspond to the final x-ray image obtained during the IVUS imaging procedure. This final x-ray image may depict the end location of the IVUS imaging device 820 during the IVUS imaging procedure. In this example, all points along the timeline 921 may represent x-ray images obtained at some point throughout the IVUS imaging procedure. The timeline 921 may be of any suitable appearance to convey to the user information relating to the time of an imaging procedure. For example, the timeline 921 could be a line, such as a solid or patterned line of any suitable color, pattern, width, or extend along any suitable path, including a linear or non-linear path. The timeline 921 may also be any suitable shape, size, color, or orientation.

The marker 922 may indicate to the user of the system 100 at what point in time during the imaging procedure the displayed x-ray image 910 was acquired. For example, if the marker 922 was positioned at the left-most position along the timeline 921, the image 910 may be the first image acquired during the procedure. If the marker 922 was positioned at the right-most position along the timeline 921, the image 910 may be the final image acquired. If the marker 922 is positioned at any position therebetween, the image 910 may be an image acquired at some point after the first image was acquired and before the final image was acquired. The marker 922 may be of any suitable appearance, including any suitable size, shape, color, or orientation.

Upon a user input selecting the button 924, the processor circuit continuously display the obtained x-ray images in the order in which they were obtained starting with the image associated with the location of the marker 922 along the timeline 921. For example, the button 924 may be a play/pause button. When a user selects the button 924, the processor circuit may continuously step through the x-ray images acquired in a chronological order. The images may be displayed at any rate of speed, including at the rate at which they were acquired or may be displayed at a faster or slower rate. In some embodiments, the user of the system 100 may adjust this rate. The user of the system 100 may again select the button 924 to pause the stream of x-ray images as needed. A still x-ray image (one of frames in the x-ray image stream) may then be displayed to the user for inspection.

The buttons 926 and 928 may allow the user of the system 100 to manually step through the acquired x-ray images. For example, the user may wish to view the x-ray image 910. Upon a user input selecting the button 924, the processor circuit may pause the stream of x-ray images and display the image 910. In response to a user input selecting the button 928, the processor circuit may display the x-ray image obtained directly after the image 910 was obtained. Similarly, in response to a user input selecting the button 926, the processor circuit may display the x-ray image obtained directly before the image 910. In some embodiments, in response to a selection of the buttons 926 and 928, the processor circuit may change the displayed x-ray image to the image obtained immediately before or after the displayed image as just described. In some embodiments, the processor circuit may display an image corresponding to any suitable number of images before or after the displayed image in response to the selection of the buttons 926 and/or 928. For example, the selection of the button 928 may not display the next image obtained immediately after the displayed image but may display the image obtained five images downstream of the displayed image. In this way, the user may manually navigate through the x-ray images more quickly. In response to a user input selecting and holding the buttons 926 and 928, the processor circuit may also cause the displayed x-ray images to move more quickly through the sequence.

As an x-ray image 910 is displayed to the user of the system 100, a marker 940 may be overlaid over the image 910. The marker 940 may be of any suitable appearance, including any shape, profile, color, pattern, or position. For example, the marker 940 may be of triangular shape shown. In other embodiments, the marker 940 may be a line or rectangle extending across the pathway 830 in a perpendicular or transverse direction. The marker 940 may also be of any other suitable appearance. The marker 940 may identify the location of the imaging device 820 within the image 910 as calculated by the system 100. The processor circuit of the system 100 may determine the location of the imaging device 820 within each acquired x-ray image. The location of the IVUS imaging device 820 may be identified within an x-ray image by a darkened area within the x-ray image because the device 820 is constructed of radiopaque material. The processor circuit may identify the radiopaque device 820 within each x-ray image by any suitable method as described with reference to FIG. 7. In the illustrated embodiment, the processor circuit has determined the correct location of the imaging device 820 and the marker 940 because the location of the marker 940 is aligned with the location of the radiopaque portion of the imaging device 820. The marker 940 may also correspond to an IVUS image. The marker 940 may indicate that the system 100 determined that the corresponding IVUS image was acquired at the location of the marker 940 along the pathway 830.

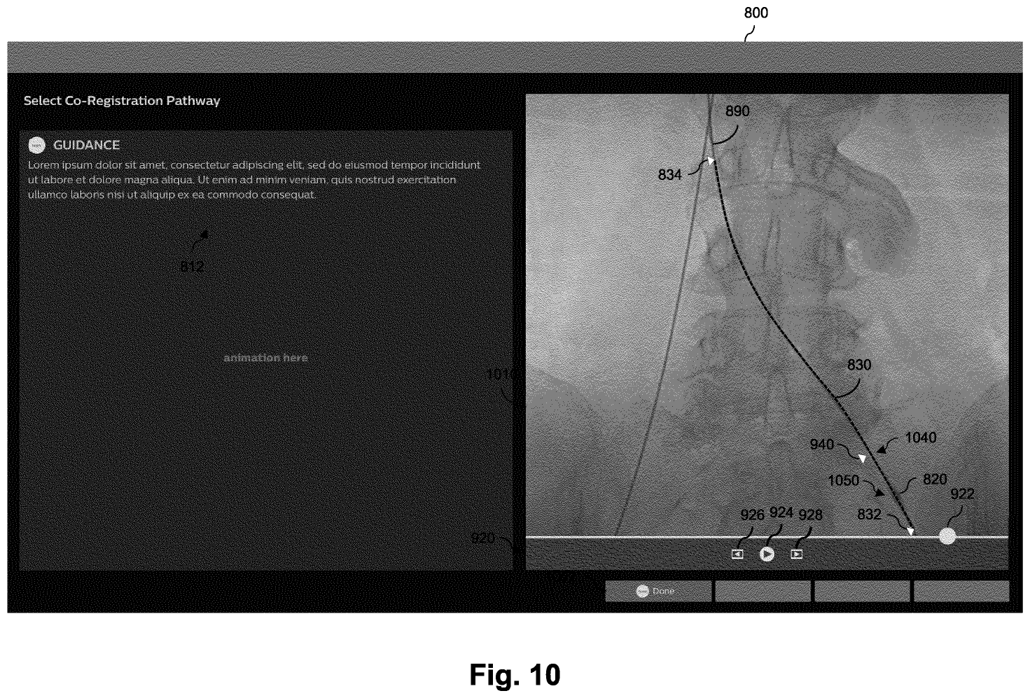

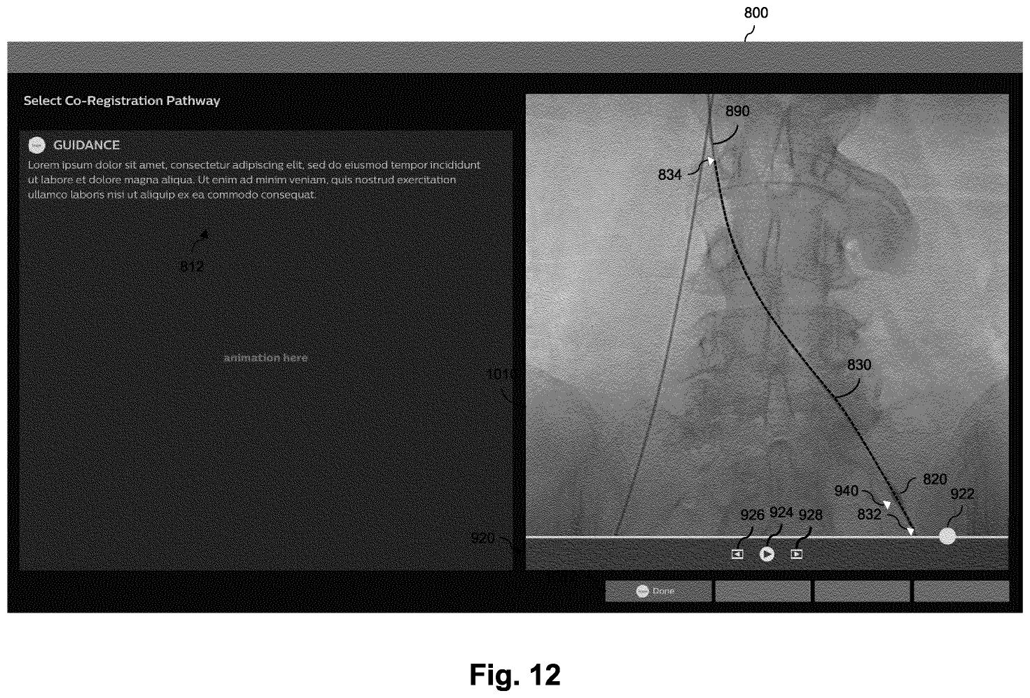

As each x-ray image is displayed to the user, the imaging device 820 may be in a different location. For example, when the user selects the button 924 to view the image stream in a video format, the radiopaque portion of the device 820 may move along the pathway 830 from the location of the indicator 832 to the location of the indicator 834. The marker 940 may also move alongside the device 820. In this way, the user of the system 100 may visually verify that the marker 940 correctly identifies the location of the device 820 throughout the imaging procedure. If, for any x-ray image shown, the marker 940 does not correspond to the correct location of the imaging device 820, the user may correct the location of the marker 940, as described with reference to FIGS. 10 and 12.

FIG. 10 is a diagrammatic view of a graphical user interface displaying a marker 940 incorrectly identifying a location of an imaging device 820 within an x-ray image 1010, according to aspects of the present disclosure. The graphical user interface 800 shown in FIG. 10 includes many of the same features described with reference to FIG. 9. The interface 800 in FIG. 10 includes an x-ray image 1010, a location 1040, a location 1050, and a button 1052.

Like the x-ray image 810 of FIG. 8 and the x-ray image 910 of FIG. 9, the x-ray image 1010 may be an image acquired by the x-ray imaging system 151 during the imaging procedure. However, the image 1010 may differ from the images 810 and 910 in that the image 1010 may have been acquired at a different time during the imaging procedure than the images 810 and 910. However, in some circumstances, two of the images 810, 910, or 1010 may be the same image. For example, the image 1010 may be the same image as the image 810 of FIG. 8.

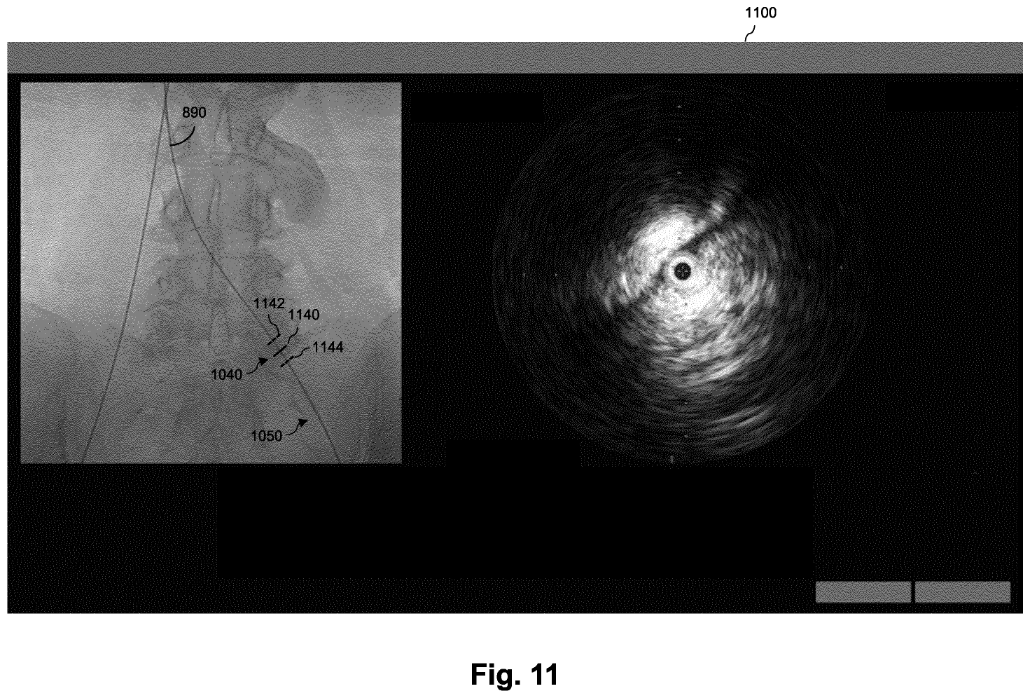

The image 1010 may represent an x-ray image in which the marker 940 incorrectly identifies the location of the imaging device 820. As shown in FIG. 10, the marker 940 is positioned at a location proximal to the radiopaque portion of the device 820. This means that the system 100 did not correctly identify the location of the device 820 within the image 1010. This also means that the IVUS image corresponding to the x-ray image 1010 is coregistered to the incorrect location along the pathway 830. Specifically, and as will be described with reference to FIG. 11, the system 100 may incorrectly indicate to the user of the system 100 that an IVUS image obtained at the location 1050 was obtained at the location 1040. If this IVUS image shows a region of interest, such as a location where the vessel is constricted or the location of a lesion, a physician may place a stent or perform any other therapy or procedure at the incorrect location.

To remedy the issue, a user of the system 100 may move the marker 940 to the correct position. For example, in one embodiment, after the processor circuit pauses the image stream in response to a user input selecting the button 924, the user may ensure that a still image 1010 is displayed using the panel 920. The user may click, tap, or otherwise indicate the location 1050 within the image 1010. The location 1050 is the actual location where the radiopaque portion of the imaging device 820 is in image 1010. In response to the user input identifying the location 1050, the processor circuit may move the marker 940 from the location 1040 to the location 1050 within the display. The process may be repeated for any x-ray image in the x-ray image stream where the marker 940 (the location computed by the processor circuit for the imaging device 820) does not match the actual location of the radiopaque portion of the imaging device 820. After the user verifies that the marker 940 is in the desired location in one or a plurality of the x-ray images, the user may then select the button 1052, which indicates that the user is done with manual adjustment to computed location(s) of the imaging device 820. In response to the selection of the button 1052, the processor circuit may then save the change(s) that the user has made to the computed location(s).

FIG. 11 is a diagrammatic view of a graphical user interface 1100 displaying an IVUS image 1130 coregistered to an incorrect location 1040 within an x-ray image 1110, according to aspects of the present disclosure. FIG. 11 shows an x-ray image 1110 and an IVUS image 1130. The x-ray image 1110 depicts the guidewire 890, includes markers 1140, 1142, and 1144 and identifies a location 1040 and a location 1050 along the guidewire 890. FIG. 11 illustrates the relationship between an exemplary IVUS image 1130 that has been incorrectly coregistered to the x-ray image 1110. In that regard, FIG. 11 can be representative of the circumstances described with respect to FIG. 10, prior to the user correction being made.

The x-ray image 1110 shown in FIG. 11 may be similar or identical to the x-ray images 810, 910, or 1010 previously described. The image 1110 may be one of many x-ray images acquired during an imaging procedure. Like images 810, 910, and 1010, the image 1110 shows the patient anatomy from the perspective of the x-ray imaging device 152 during an imaging procedure and shows the radiopaque guidewire 890. In some embodiments, the image 1110 may include a view of the imaging device 820 or may not.

The IVUS image 1130 may be an image similar to the image 730 described with reference to FIG. 7. The IVUS image 1130 may be one of the images obtained by the intravascular device 820 during an imaging procedure. With reference to FIG. 8, it may be an IVUS image obtained as the device 820 moves from the starting location 832 to the ending location 834 or moved in an opposite direction.

As explained with reference to FIG. 10, due to various factors such as the device 820 being moved too quickly or other errors, IVUS images, such as the IVUS image 1130, may not be correctly coregistered or associated with the correct locations within an x-ray image. This may be identified by the user by viewing the marker 940 (FIG. 10) not matching up with the location of the imaging device 820 as observed in an x-ray image. The location of the marker 940 (FIG. 10) corresponds to the location at which the system 100 estimates that an IVUS image was obtained. Similarly, the marker 1140 shown in FIG. 11 identifies the location 1040 at which the system 100 determined the IVUS image 1130 was obtained. Within the graphical user interface 800 shown in FIG. 10, this may correspond to the location of the marker 940 previously described.

Markers 1142 and 1144 are shown on either side of the marker 1140. Like the marker 1140, markers 1142 and 1144 may identify locations at which the system 100 determined that other IVUS images were obtained. For example, just as the marker 1140 corresponds to the determined location 1040 of the IVUS image 1130 shown, the marker 1142 may correspond to the determined location of an IVUS image obtained immediately after the IVUS image 1130 was obtained. Similarly, the marker 1144 may correspond to the determined location of an IVUS image obtained before the IVUS image 1130 was obtained. It will be appreciated that each IVUS image obtained during an imaging procedure could have a corresponding marker similar to the markers 1140, 1142, and 1144 placed at their appropriate locations along the guidewire 890. These markers, including the markers 1140, 1142, and 1144, may or may not displayed to a user of the system 100. In some instances, only one of the markers 1140, 1142, and 1144 is displayed at a time, along with the corresponding IVUS image. In some instances, multiple ones of the markers 1140, 1142, and 1144, along with the corresponding IVUS images, are displayed simultaneously.

As noted, the location 1040 of the marker 1140 on the x-ray image may not be the actual location along the guidewire 890 where the IVUS image 1130 was obtained. In other words, the location 1040 may be incorrect. Similarly, the locations of the markers 1142 and 1144, as well as other nearby markers, may also be incorrect. The correct location 1050 may be identified as described with reference to FIG. 10.

FIG. 12 is a diagrammatic view of a graphical user interface 800 displaying a marker 940 moved to the correct location of an imaging device 820 within an x-ray image 1010, according to aspects of the present disclosure. FIG. 12 may represent a display shown to the user after the location of the marker 940 has been corrected so that the marker 940 is aligned or co-located with the radiopaque portion of the imaging device 820. The location of the marker 940 may be corrected in any suitable way. For example, as described with reference to FIG. 10, after clicking, tapping, or otherwise engaging the button 852 of FIG. 8 and using the panel 920 to navigate to the x-ray image 1010, the processor circuit may move the marker 940 in response to a click on a location along the pathway 830. The user input may include clicking on the marker 940 and dragging it to the desired location. The processor circuit may move the marker 940 in response to any suitable user input identifying the correct location within the image 1010 along the pathway 830.

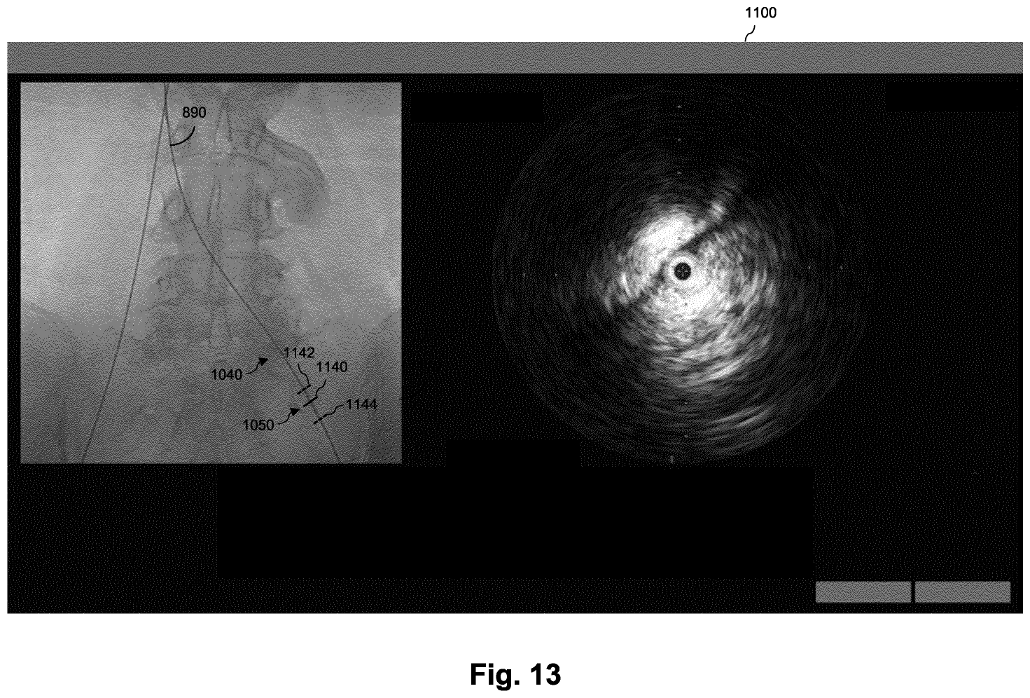

After the marker 940 has been moved to the correct location along the pathway 830, the processor circuit may save the change in location in response to a user input selecting the button 1052 or by another user input otherwise indicating that the marker 940 is in its correct location. The change may be saved by storing the new location for the imaging device 820 and/or reassigning the IVUS image or other IVUS data point corresponding to the marker 940 to a different location, as will be discussed in further detail with reference to FIG. 13. After the location of the marker 940 is confirmed, the system 100 may coregister or re-coregister all of the IVUS images received by the device 820 with their corresponding locations along the pathway 830 again. During this additional coregistration process, the locations of any other IVUS images may be modified based on the change in location of the IVUS image associated with the marker 940.

FIG. 13 is a diagrammatic view of a graphical user interface 1100 displaying an IVUS image 1130 coregistered to its correct location 1050 within an x-ray image 1110, according to aspects of the present disclosure. FIG. 13 illustrates the relationship between the exemplary IVUS image 1130 after it has been correctly coregistered to the corresponding x-ray image 1110.

After the marker 940 (FIG. 10) is moved to its correct location, the processor circuit may recalculate the coregistration of all IVUS images with locations along the pathway 830. After this recalculation, any of the locations at which IVUS images were determined by the system 100 to be acquired may be changed as result of moving the marker 940. FIG. 13 shows the result of such a recalculation. The marker 1140 shown in FIG. 13 identifies the corrected location 1050 for the marker 940. This location is the actual location at which the imaging device 820 was located when the IVUS image 1130 was acquired.

The markers 1142 and 1144 are still shown on either side of the marker 1140 in FIG. 13. As explained with reference to FIG. 11, the markers 1142 and 1144 may identify locations at which the system 100 determined the IVUS image preceding and following the IVUS image 1130 were obtained. Based on the change in the location of the marker 1140 from the incorrect location 1040 to the correct location 1050, the system 100 may adjust its estimation of the correct locations of the markers 1142 and 1144 as well.

Referring again to FIG. 12, after the location of the marker 940 has been updated by the processor circuit and the processor circuit has recalculated determined locations of all IVUS data along the pathway 830, the processor circuit may output to the display the x-ray images in sequence again allowing a user to verify that the location of the marker 940 is now correct throughout the imaging procedure. In response to user inputs of the panel 920, the processor circuit may step through all of the acquired x-ray images allowing a user to observe the location of the radiopaque portion of the imaging device 820 as it moves along the pathway 830. If the marker 940 correctly identifies the location of the device 820 through all of the x-ray images, the IVUS data is co-registered to correct locations along the pathway 830. If, for any x-ray image, the marker 940 does not correctly identify the location of the imaging device 820, like the image 1010 of FIG. 10, the processor circuit may adjust the location of the marker 940 for these x-ray images in response to a user input as described and the processor circuit may recalculate the locations of IVUS data along the pathway 830. This process of adjusting the marker 940 and recalculating coregistration may continue until the user is satisfied that the coregistration is as accurate as desired.

In some embodiments, when the user is satisfied with the coregistration of data as shown by the pathway 830 and location of the marker 940 in each acquired x-ray image, the processor circuit may store the data and no longer display the panel 920 in response to a user input selecting the button 1052 (FIG. 12). In some embodiments, referring back to FIG. 8, when the user is satisfied with the coregistration of data, the user may additionally or alternative use the button 856 to confirm the pathway. In other embodiments, the user may indicate to the system 100 that the pathway 830 is in a satisfactory state by any other suitable means. At this point, the system 100 may save the data relating to the updated coregistration within any suitable storage location in communication with the system 100. This data may include any x-ray images or IVUS images acquired, the pathway 830, markers or data indicating the locations of IVUS data along the pathway 830 or within x-ray images in general, notes or annotations of the user, or any other suitable data.

FIG. 14 is a diagrammatic view of a graphical user interface 1400 displaying a coregistration pathway 1430 with a portion that does not have the same shape as guidewire 890, according to aspects of the present disclosure. The graphical user interface 1400 includes the x-ray image 810. Overlaid on the x-ray image 810, FIG. 14 depicts a pathway 1430 including an indicator 1432, an indicator 1434, and multiple nodes 1440. FIG. 14 also identifies a location 1450.

In some embodiments, the processor circuit may display the graphical user interface 1400 to a user in response to a user input selecting the button 854 (FIG. 8). The graphical user interface 1400 may be similar to the interface 800 and/or the interface 1100 described previously. The processor circuit may allow the user to correct the shape of the pathway 1430 overlaid on the x-ray image 810 so it matches the shape of the guidewire 890.

The pathway 1430 may be calculated by the processor circuit of the system 100 during coregistration after the IVUS imaging procedure using the locations of the radiopaque portions of the imaging device in the x-ray images. The pathway 1430 may be similar to the pathway 830 of FIG. 8. In an exemplary procedure, a processor circuit may receive multiple x-ray images and IVUS images obtained during an IVUS imaging procedure. As described with reference to the pathway 830 of FIG. 8, the IVUS images may be coregistered to appropriate positions along the pathway 1430. The location of the marker 940 may be adjusted by the processor circuit in response to user inputs for any of the received x-ray images to correct the location of any IVUS images along the pathway 1430 as necessary as previously described. In some embodiments, however, the shape of the pathway 1430 may not align with the shape of the guidewire 890 as shown in FIG. 14. In that regard, because the imaging catheter was moved along the guidewire during the IVUS imaging acquisition, the shape of the pathway should match the shape of the guidewire. For example, at one or more locations along the pathway 1430, the pathway 1430 may not be overlaid over the guidewire 890. For one or more locations, there may be some distance, space, or gap between the pathway 1430 and the guidewire 890. The processor circuit may allow a user to correct these areas where the pathway 1430 and the guidewire 890 do not align with the graphical user interface 1400 as will be described. The pathway 1430 may not align with the guidewire 890 for various reasons. For example, the system 100 may not have correctly identified the locations of the radiopaque portions of the imaging device or the guidewire 890 during coregistration.

The system 100 may display to the user the pathway 1430 overlaid over an x-ray image. The pathway 1430 may include an indicator 1432 and an indicator 1434. These indicators may denote the starting and stopping locations of the intravascular device during movement through the vessel. For example, the indicator 1432 may show a starting location and the indicator 1434 may show an ending location. In some embodiments, the indicator 1434 may be a starting location and the indicator 1432 may be an ending location. The indicators 1432 and 1434 may be of any suitable appearance, including of any suitable shape, color, pattern, or orientation.

Along the pathway 1430, multiple nodes 1440 are shown. The nodes may be displayed in the screen display after the button 854 (FIG. 8) is clicked by the user. In some embodiments, these nodes may represent locations at which IVUS images were obtained. In some embodiments, these nodes do not necessarily represent locations at which IVUS images are obtained but are generated by the system 100 along the pathway 1430 and spaced apart in a uniform way. The system 100 may place nodes 1440 along the pathway 1430 according to any particular pattern. For example, a node 1440 may be equally spaced along the pathway 1430. In other embodiments, the system 100 may generate nodes 1440 and place them on the pathway 1430 based on the shape or contours of the pathway 1430. In some embodiments, a user of the system 100 may be able to add or remove nodes 1440 at any suitable location along the pathway 1430.

In response to a user input, the processor circuit may alter the shape of the pathway 1430 by moving any of the nodes 1440. As an example, at a location 1450 along the guidewire 890, the pathway 1430 deviates from the shape of the guidewire 890. The pathway 1430 may be generated by the processor circuit based on the locations of the radiopaque portion of the intravascular device 820 in the acquired x-ray images and the detected location of the guidewire 890. The location of the radiopaque portion of the intravascular device 820 may be determined by image processing or other suitable techniques. Because the intravascular device 820 is positioned on the guidewire 890 such that the guidewire extends through a lumen of the device 820, the intravascular device 820 only moves along the guidewire within the patient anatomy. The deviation shown at the location 1450 may, therefore, indicate that the processor circuit incorrectly determined the location of the device 820 at that location in the corresponding x-ray images.

To adjust the shape of the pathway 1430, the processor circuit may move any of the nodes 1440 within the image 810 in response to a user input. The user input may be a click, a click and drag, a touch on a touch screen, or any other suitable input. For example, to remedy the discrepancy in shape between the pathway 1430 and guidewire 890 at the location 1450, the user may select and move the node 1442. In response to the input, the processor circuit may move the node 1442 to the location of the guidewire 890 at the location 1450.

FIG. 15 is a diagrammatic view of a graphical user interface 1400 displaying a coregistration pathway 1430 that has been corrected to have the same shape as a guidewire 890, according to aspects of the present disclosure. After the node 1442 is moved to the correct location, the system 100 may recalculate the coregistration of all IVUS images with locations along the pathway 830. After this recalculation, any of the locations at which IVUS images were determined by the system 100 to be acquired may be edited. FIG. 15 shows the result of such a recalculation. The node 1442 shown in FIG. 15 is shown to be moved to its correct location along the guidewire 890.

One or a plurality of other nodes 1440 may also be adjusted based on the correction of the node 1442. Based on the change in the location of the node 1442 from its incorrect location to the correct location, the system 100 may adjust its estimation of the correct locations of all or some of the other nodes 1440. As each node 1440 is adjusted, the locations of acquired IVUS images determined to have been acquired at locations around that node 1440 may also be adjusted.

After the processor circuit has updated the location of the node 1442 and recalculated determined locations of all or some IVUS data along the pathway 1430, the shape of the pathway 1430 may be verified to align with the shape of the guidewire 890 within the x-ray image. If the pathway 1430 aligns with the shape of the guidewire 890, the coregistration of IVUS data is coregistered to correct locations along the pathway 1430. If the pathway 1430 does not align with the shape of the guidewire 890, the location of other nodes 1440 may be adjusted and the processor circuit may recalculate the locations of IVUS data along the pathway 1430 as has been described. This process of adjusting the nodes 1440 and recalculating coregistration may continue until the user is satisfied that the coregistration is as accurate as desired.

When the user is satisfied with the shape of the pathway 1430, the user may select the button 1452. In other embodiments, referring back to FIG. 8, the user may additionally or alternatively use the button 856 (FIG. 8) to confirm the pathway. In other embodiments, the user may indicate to the system 100 that the shape of the pathway 1430 is satisfactory by any other suitable means. At this point, the system 100 may save the data relating to coregistration within any suitable storage location in communication with the system 100. This data may include any x-ray images or IVUS images acquired, the pathway 1430, markers or data indicating the locations of IVUS data along the pathway 1430 or within x-ray images in general, notes or annotations of the user, or any other suitable data. It is noted that in some embodiments, the system 100 may perform one, the other, or both of correcting the location of the radiopaque portion of the intravascular device within an x-ray image as described with reference to FIGS. 9-13 and correcting the pathway overlaid over an x-ray image as described with reference to FIGS. 14-15. In some embodiments, the system 100 only corrects the identified location of the radiopaque portion of the intravascular device along the guidewire or corrects the pathway overlaid on an x-ray image. In some embodiments, the system 100 may perform both functions.

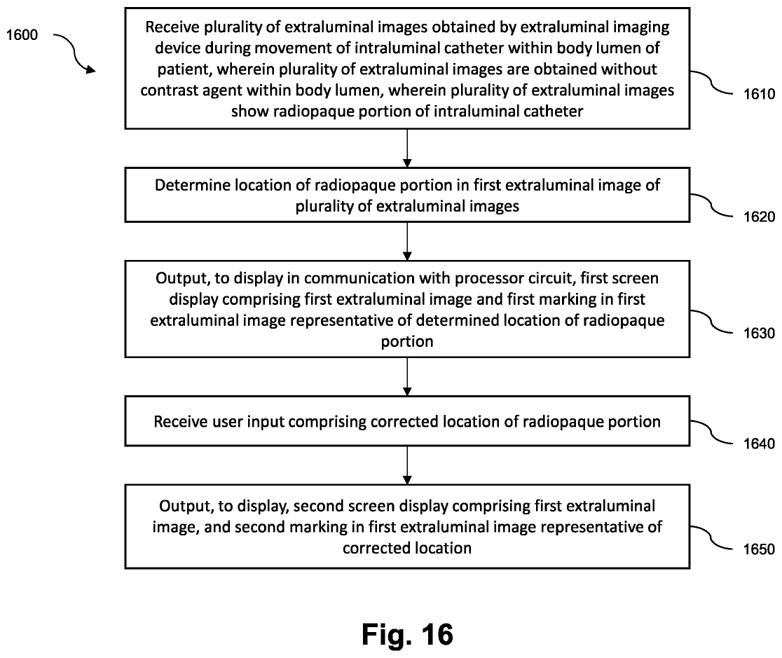

FIG. 16 is a flow diagram for a method of modification of the location of an intraluminal imaging device within an extraluminal image, according to aspects of the present disclosure. As illustrated, the method 1600 includes a number of enumerated steps, but embodiments of the method 1600 may include additional steps before, after, or in between the enumerated steps. In some embodiments, one or more of the enumerated steps may be omitted, performed in a different order, or performed concurrently. The steps of the method 1600 can be carried out by any suitable component within the system 100 and all steps need not be carried out by the same component. In some embodiments, one or more steps of the methods 1600 can be performed by, or at the direction of, a processor circuit of the system 100 (e.g., the processor circuit 510 of FIG. 5), including, e.g., the processor 560 or any other component.

At step 1610, the method 1600 includes receiving a plurality of extraluminal images obtained by the extraluminal imaging device during movement of an intraluminal catheter within a body lumen of a patient. In some embodiments, the plurality of extraluminal images are obtained without a contrast agent within the body lumen. In some embodiments, the plurality of extraluminal images are obtained with a contrast agent within the body lumen. The plurality of extraluminal images show a radiopaque portion of the intraluminal catheter. For example, step 1610 can include receiving a plurality of x-ray images obtained by the x-ray imaging device during movement of the IVUS imaging catheter within a blood vessel of a patient. In some embodiments, the plurality of x-ray images are obtained without a contrast agent within the blood vessel. In some embodiments, the plurality of x-ray images are obtained with a contrast agent within the blood vessel. The plurality of x-ray images show a radiopaque portion of the IVUS imaging catheter.

At step 1620, the method 1600 includes determining a location of the radiopaque portion in a first extraluminal image of the plurality of extraluminal images. For example, step 1620 can include receiving a plurality of IVUS images obtained by the IVUS imaging catheter during the movement of the IVUS imaging catheter, determining a path of the movement based on corresponding locations of the radiopaque portion in the plurality of x-ray images, and co-registering the plurality of IVUS images to corresponding positions along the path.

At step 1630, the method 1600 includes outputting, to a display in communication with the processor circuit, a first screen display comprising the first extraluminal image, and a first marking in the first extraluminal image representative of the determined location of the radiopaque portion. For example, step 1630 can include outputting, to a display in communication with the processor circuit, a first screen display comprising the plurality of x-ray images, and a first marking in each x-ray image of the plurality of x-ray images representative of the corresponding location of the radiopaque portion such that the first marking is shown at different positions corresponding to the movement of the intraluminal catheter receive a user input comprising a corrected location of the radiopaque portion.

At step 1640, the method 1600 includes receiving a user input comprising a corrected location of the radiopaque portion. For example, the step 1640 can include receiving a user input comprising a corrected location of the radiopaque portion in a first x-ray image of the plurality of x-ray images.

At step 1650, the method 1600 includes outputting, to the display, a second screen display comprising the first extraluminal image, and a second marking in the first extraluminal image representative of the corrected location. For example, the step 1650 can include determining a corrected position along the determined path for a corresponding IVUS image based on the user input comprising the corrected location of the radiopaque portion; and outputting, to the display, a second screen display comprising a second x-ray image of the plurality of x-ray images, the corresponding IVUS image, and a second marking in the second x-ray image representative of the corrected position along the path.

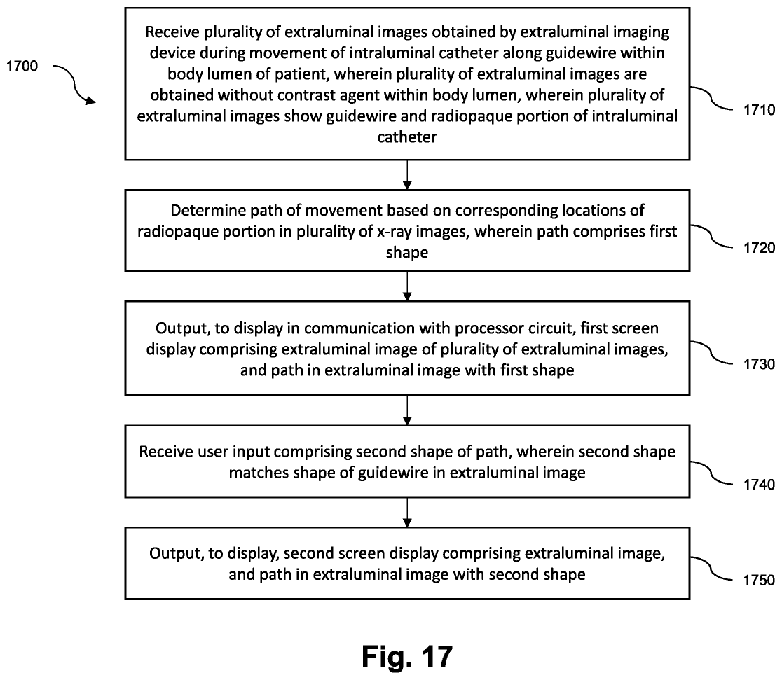

FIG. 17 is a flow diagram for a method 1700 of pathway modification for coregistration of an extraluminal image and intraluminal data, according to aspects of the present disclosure. As illustrated, the method 1700 includes a number of enumerated steps, but embodiments of the method 1700 may include additional steps before, after, or in between the enumerated steps. In some embodiments, one or more of the enumerated steps may be omitted, performed in a different order, or performed concurrently. The steps of the method 1700 can be carried out by any suitable component within the system 100 and all steps need not be carried out by the same component. In some embodiments, one or more steps of the methods 1700 can be performed by, or at the direction of, a processor circuit of the system 100 (e.g., the processor circuit 510 of FIG. 5), including, e.g., the processor 560 or any other component.

At step 1710, the method 1700 includes receiving a plurality of extraluminal images obtained by the extraluminal imaging device during movement of an intraluminal catheter along a guidewire within a body lumen of a patient. In some embodiments, the plurality of extraluminal images are obtained without a contrast agent within the body lumen and the plurality of extraluminal images show the guidewire and a radiopaque portion of the intraluminal catheter. For example, the step 1710 can include receiving a plurality of x-ray images obtained by the x-ray imaging device during movement of the IVUS imaging catheter along a guidewire within a blood vessel of a patient. In some embodiments, the plurality of x-ray images are obtained without a contrast agent within the blood vessel and the plurality of extraluminal images show the guidewire and a radiopaque portion of the IVUS catheter.

At step 1720, the method 1700 includes determining a path of the movement based on corresponding locations of the radiopaque portion in the plurality of x-ray images. The path may comprise a first shape. For example, the step 1720 can include receiving a plurality of IVUS images obtained by the IVUS imaging catheter during the movement of the IVUS imaging catheter and determining a path of the movement based on corresponding locations of the radiopaque portion in the plurality of x-ray images. The path may comprise a first shape.

At step 1730, the method 1700 includes outputting, to a display in communication with the processor circuit, a first screen display comprising an extraluminal image of the plurality of extraluminal images and the path in the extraluminal image with the first shape. For example, the step 1730 can include outputting, to a display in communication with the processor circuit, a first screen display comprising an x-ray image of the plurality of x-ray images and the path in the x-ray image with the first shape.

At step 1740, the method 1700 includes receiving a user input comprising a second shape of the path. In some embodiments, the second shape matches a shape of the guidewire in the extraluminal image. For example, the step 1740 can include receiving a user input comprising a second shape of the path. In some embodiments, the second shape matches a shape of the guidewire in the x-ray image.

At step 1750, the method 1700 includes outputting, to the display, a second screen display comprising the extraluminal image and the path in the extraluminal image with the second shape. For example, the step 1750 can include co-registering the plurality of IVUS images to positions along the path with the second shape, outputting, to the display, a second screen display comprising a second x-ray image of the plurality of x-ray images, an IVUS image, and a marking in the x-ray image representative a corresponding position of the IVUS image along the path with the second shape.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a processor circuit configured for communication with an extraluminal imaging device, wherein the processor circuit is configured to:
receive a plurality of extraluminal images obtained by the extraluminal imaging device during movement of an intraluminal catheter within a body lumen of a patient, wherein the plurality of extraluminal images show a radiopaque portion of the intraluminal catheter;
determine a location of the radiopaque portion in a first extraluminal image of the plurality of extraluminal images;
output, to a display in communication with the processor circuit, a first screen display comprising:
the first extraluminal image; and
a first marking in the first extraluminal image representative of the determined location of the radiopaque portion;
receive a user input comprising a corrected location of the radiopaque portion; and
output, to the display, a second screen display comprising:
the first extraluminal image; and
a second marking in the first extraluminal image representative of the corrected location.

2. The system of claim 1, wherein the processor circuit is configured to:
determine a corresponding location of the radiopaque portion in each extraluminal image of a plurality of extraluminal images;
output, to the display, the plurality of extraluminal images such that a respective extraluminal image in the first screen display includes the first marking representative of the corresponding location of the radiopaque portion.

3. The system of claim 2, wherein the plurality of extraluminal images show the radiopaque portion at different positions corresponding to the movement of the intraluminal catheter.

4. The system of claim 2, wherein the plurality of extraluminal images show the first marking at different positions corresponding to the movement of the intraluminal catheter.

5. The system of claim 2, wherein the processor circuit is configured to receive a further user input comprising a selection of the first extraluminal image from among the plurality of extraluminal images.

6. The system of claim 2, wherein the processor circuit is configured to:
output, to the display, the plurality of extraluminal images such that the first extraluminal image in the second screen display includes the second marking representative of the corrected location.

7. The system of claim 2,
wherein the processor circuit is configured to determine a path of the movement based on the corresponding locations of the radiopaque portion in the plurality of extraluminal images,
wherein the first extraluminal image in first screen display further includes the determined path.

8. The system of claim 7,
wherein the intraluminal catheter comprises an intraluminal imaging catheter,
wherein the processor circuit is configured for communication with the intraluminal imaging catheter,
wherein the processor circuit is configured to:
receive a plurality of intraluminal images obtained by the intraluminal imaging catheter during the movement of the intraluminal imaging catheter; and
coregister the plurality of intraluminal images to corresponding positions along the determined path.

9. The system of claim 8,
wherein the processor circuit is configured to determine a corrected position along the determined path for a corresponding intraluminal image based on the user input comprising the corrected location of the radiopaque portion.

10. The system of claim 9, wherein the processor circuit is configured to output, to the display, a third screen display comprising:
a second extraluminal image of the plurality of extraluminal images;
the corresponding intraluminal image; and
a third marking in the extraluminal image representative of the corrected position of the intraluminal image along the path.

11. The system of claim 8,
wherein the processor circuit is configured to further coregister the plurality of intraluminal images to corresponding positions along the determined path based on the user input comprising the corrected location of the radiopaque portion.

12. The system of claim 7,
wherein the movement of the intraluminal catheter is along a guidewire within the body lumen,
wherein the plurality of extraluminal images further show the guidewire,
wherein the path matches the shape of the guidewire within the body lumen.

13. The system of claim 1, wherein the plurality of extraluminal images are obtained without a contrast agent within the blood vessel.

14. A system, comprising:
an intravascular ultrasound (IVUS) imaging catheter; and
a processor circuit configured for communication with an x-ray imaging device and the IVUS catheter, wherein the processor circuit is configured to:
receive a plurality of x-ray images obtained by the x-ray imaging device during movement of the IVUS imaging catheter within a blood vessel of a patient, wherein the plurality of x-ray images are obtained without a contrast agent within the blood vessel, wherein IVUS plurality of extraluminal images show a radiopaque portion of the intraluminal catheter;
receive a plurality of IVUS images obtained by the IVUS imaging catheter during the movement of the IVUS imaging catheter;
determine a path of the movement based on corresponding locations of the radiopaque portion in the plurality of x-ray images;
co-register the plurality of IVUS images to corresponding positions along the path;
output, to a display in communication with the processor circuit, a first screen display comprising:
the plurality of x-ray images; and
a first marking in each x-ray image of the plurality of x-ray images representative of the corresponding location of the radiopaque portion such that the first marking is shown at different positions corresponding to the movement of the intraluminal catheter;
receive a user input comprising a corrected location of the radiopaque portion in a first x-ray image of the plurality of x-ray images;
determine a corrected position along the determined path for a corresponding IVUS image based on the user input comprising the corrected location of the radiopaque portion; and
output, to the display, a second screen display comprising:
a second x-ray image of the plurality of x-ray images;
the corresponding IVUS image; and
a second marking in the second x-ray image representative of the corrected position along the path.

15. A system, comprising:
a processor circuit configured for communication with an extraluminal imaging device, wherein the processor circuit is configured to:
receive a plurality of extraluminal images obtained by the extraluminal imaging device during movement of an intraluminal catheter along a guidewire within a body lumen of a patient, wherein the plurality of extraluminal images are obtained without a contrast agent within the body lumen, wherein the plurality of extraluminal images show the guidewire and a radiopaque portion of the intraluminal catheter;
determine a path of the movement based on corresponding locations of the radiopaque portion in the plurality of extraluminal images, wherein the path comprises a first shape;
output, to a display in communication with the processor circuit, a first screen display comprising:
an extraluminal image of the plurality of extraluminal images; and
the path in the extraluminal image with the first shape;
receive a user input comprising a second shape of the path, wherein the second shape matches a shape of the guidewire in the extraluminal image; and
output, to the display, a second screen display comprising:
the extraluminal image; and
the path in the extraluminal image with the second shape.

16. A system, comprising:
an intravascular ultrasound (IVUS) imaging catheter; and
a processor circuit configured for communication with an x-ray imaging device and the IVUS catheter, wherein the processor circuit is configured to:
receive a plurality of x-ray images obtained by the x-ray imaging device during movement of the IVUS imaging catheter along a guidewire within a blood vessel of a patient, wherein the plurality of x-ray images are obtained without a contrast agent within the blood vessel, wherein the plurality of extraluminal images show the guidewire and a radiopaque portion of the IVUS catheter;
receive a plurality of IVUS images obtained by the IVUS imaging catheter during the movement of the IVUS imaging catheter;
determine a path of the movement based on corresponding locations of the radiopaque portion in the plurality of x-ray images, wherein the path comprises a first shape;
output, to a display in communication with the processor circuit, a first screen display comprising:
an x-ray image of the plurality of x-ray images; and
the path in the x-ray image with the first shape;
receive a user input comprising a second shape of the path, wherein the second shape matches a shape of the guidewire in the x-ray image;
co-register the plurality of IVUS images to positions along the path with the second shape; and
output, to the display, a second screen display comprising:
a second x-ray image of the plurality of x-ray images;
an IVUS image; and
a marking in the x-ray image representative a corresponding position of the IVUS image along the path with the second shape.
